(19) United States
(12) Patent Application Publication
(10) Pub. No.: US 2023/0083456 A1
(43) Pub. Date: Mar. 16, 2023

(54) COMPOSITIONS AND METHODS FOR DIAGNOSING COLORECTAL CANCER

(71) Applicant: GeneGenieDx Corporation, San Jose, CA (US)

(72) Inventors: Wenying Pan, Los Gatos, CA (US); Xiao Yang, Dublin, CA (US)

(21) Appl. No.: 17/930,460

(22) Filed: Sep. 8, 2022

Related U.S. Application Data
(60) Provisional application No. 63/241,540, filed on Sep. 8, 2021.

(51) Int. Cl. C12Q 1/6886; C12Q 1/689; G16B 30/00

(52) U.S. Cl. CPC C12Q 1/6886; C12Q 1/689; G16B 30/00; C12Q 2600/158

(57) ABSTRACT

The present disclosure provides methods and compositions, e.g., kits, for diagnosing colorectal cancer or advanced colorectal adenoma based on a subject's gut microbial markers.

### CROSS-REFERENCE TO RELATED APPLICATIONS

This application claims the priority of U.S. provisional application No. 63/241,540, filed Sep. 8, 2021, the entire disclosure of which is incorporated herein by reference.

### SEQUENCE LISTING

The sequence listing that is contained in the file named “SEQUENCE LISTING”, which is 54,695 bytes and was created on Sep. 8, 2022, is filed herewith by electronic submission and is incorporated by reference herein.

### FIELD OF THE DISCLOSURE

The present disclosure generally relates to cancer diagnosis, prognosis and treatment. In particular, the present disclosure relates to bacterial biomarkers in a feces sample for diagnosing and prognosing colorectal cancer and advanced colorectal adenoma.

### BACKGROUND

Colorectal cancer (CRC), also known as colon cancer or rectal cancer, is a cancer developed from the colon or rectum. Globally, CRC is the third most common type of cancer, making up about 10% of all cases. In 2018, there were 1.09 million new cases and over five hundred thousand deaths from the disease.

Colonoscopy is the endoscopic examination of the large bowel and the distal part of the small bowel with a CCD camera or a fiber optic camera on a flexible tube passed through the anus. It can provide a visual diagnosis (e.g., ulceration, polyps) and grants the opportunity for biopsy or removal of suspected colorectal cancer lesions. Colonoscopy is considered the “gold standard” for colon cancer diagnosis which has high sensitivity for adenoma (polyps ≥10 mm, 90% sensitivity) and carcinoma (95% sensitivity). It can remove polyps during the procedure to reduce the risk of turning to cancer, and the removed polyps can be checked to confirm if they are precancerous/cancerous by tissue diagnosis. However, colonoscopy is an invasive procedure, usually performed with conscious or deep sedation and there may be serious risks, such as serious bleeding, bowel perforation, or cardiopulmonary events.

Several tests have been developed based on feces samples, including fecal occult blood test, fecal immunochemical test, fecal DNA test, and gut microbial test.

Fecal occult blood test is designed to evaluate fecal samples for hidden blood by detecting the heme part of hemoglobin, which can be an early sign of polyps and cancer. Bleeding from other sources, such as hemorrhoids, ulcers and inflammatory bowel disease may interfere with the test to give rise to false positive results. The test may also give rise to false-negative results if the cancer or polyps do not bleed during the time the sample is taken.

Fecal immunochemical test (FIT) is also designed to detect hidden blood in fecal samples but via globin of hemoglobin. FIT is user-friendly and relatively inexpensive. However, FIT has relatively low sensitivity and may also give rise to false positive results caused by hemorrhoids, ulcers and inflammatory bowel disease.

Multi-target fecal DNA test detects certain DNA markers (mutations)in feces samples that are associated with colon neoplasia. The test has relatively higher sensitivity compared to FIT. However, the specificity of the fecal DNA test is relatively low with more false-positive rate than FIT.

Gut microbial test detects specific gut microbial markers in feces samples that are associated with colon neoplasia. Mounting evidence from metagenomic analyses suggests that a state of pathological microbial imbalance or dysbiosis is prevalent in the gut of patients with colorectal cancer. Several bacterial taxa have been identified of which representative isolate cultures interact with human cancer cells in vitro and trigger disease pathways in animal models. However, most of the current gut microbial tests depend on the sequencing of 16S rRNA gene and often identify only the genus level. On the other hand, whole genome sequencing (WGS) allows for more accurate detection of species but is much more expensive and time consuming for analysis.

Therefore, there is a continuing need to develop new tests for diagnosing, prognosing and treating colorectal cancer and advanced colorectal adenoma.

### SUMMARY OF DISCLOSURE

The present disclosure in one aspect provides a method for diagnosing colorectal cancer or advanced colorectal adenoma in a subject. In some embodiments, the method comprises: measuring in a feces sample isolated from the subject levels of at least two bacterial markers selected from the group consisting of Fusobacterium nucleatum, Peptostreptococcus stomatis, Parvimonas micra, Gemella morbillorum, Solobacterium moorei, Porphyromonas asaccharolytica, Peptostreptococcus anaerobius, Hungatella hathewayi, Streptococcus gallolyticus, Clostridium symbiosum, Prevotella copri, Prevotella nigrescens, Bacteroides clams, genotoxic pks+Escherichia coli and gene bft from Bacteroides fragilis, evaluating the measured levels of the bacterial markers, and determining that the subject is healthy or has colorectal cancer or advanced colorectal adenoma.

In some embodiments, the measured levels of the bacterial markers are evaluated by a machine learning classifier.

In some embodiments, the method comprises measuring levels of the two bacterial markers illustrated in anyone of the following groups (1)-(6): (1) Peptostreptococcus stomatis and Parvimonas micra; (2) Peptostreptococcus stomatis and Fusobacterium nucleatum; (3) Peptostreptococcus stomatis and Gemella morbillorum; (4) Fusobacterium nucleatum and Parvimonas micra; (5) Gemella morbillorum and Parvimonas micra; (6) Fusobacterium nucleatum and Gemella morbillorum.

In some embodiments, the method comprises measuring levels of at least three bacterial markers selected from the group disclosed above. In some embodiments, the method comprises measuring levels of at least three bacterial markers selected from the group consisting of Fusobacterium nucleatum, Peptostreptococcus stomatis, Parvimonas micra, Gemella morbillorum, Solobacterium moorei, and Clostridium symbiosum. In some embodiments, the method comprises measuring levels of the three bacterial markers illustrated in anyone of the following groups (1)-(13):
(1) Peptostreptococcus stomatis, Parvimonas micra, Clostridium symbiosum;
(2) Peptostreptococcus stomatis, Parvimonas micra, Fusobacterium nucleatum;
(3) Peptostreptococcus stomatis, Parvimonas micra, Solobacterium moorei;
(4) Peptostreptococcus stomatis, Parvimonas micra, Gemella morbillorum;
(5) Peptostreptococcus stomatis, Clostridium symbiosum, Fusobacterium nucleatum;
(6) Peptostreptococcus stomatis, Clostridium symbiosum, Gemella morbillorum;
(7) Peptostreptococcus stomatis, Solobacterium moorei, Gemella morbillorum;
(8) Parvimonas micra, Clostridium symbiosum, Fusobacterium nucleatum;
(9) Parvimonas micra, Clostridium symbiosum, Solobacterium moorei;
(10) Parvimonas micra, Clostridium symbiosum, Gemella morbillorum;
(11) Parvimonas micra, Fusobacterium nucleatum, Solobacterium moorei;
(12) Parvimonas micra, Fusobacterium nucleatum, Gemella morbillorum;
(13) Parvimonas micra, Solobacterium moorei, Gemella morbillorum

In some embodiments, the levels of the bacterial markers are measured via ddPCR or qPCR.

In some embodiments, measuring the levels of the bacterial markers comprises detecting a sequence selected from SEQ ID NOs: 4, 8, 12, 16, 20, 24, 28, 32, 36, 40, 44, 48, 52, 56, 60.

In some embodiments, the method comprises measuring levels of at least four bacterial markers selected from the group disclosed above. In some embodiments, the method comprises measuring levels of at least four bacterial markers selected from the group consisting of Fusobacterium nucleatum, Peptostreptococcus stomatis, Parvimonas micra, Gemella morbillorum, Solobacterium moorei, and Clostridium symbiosum. In some embodiments, the method comprises measuring levels of the following four bacterial markers:
(1) Fusobacterium nucleatum, Gemella morbillorum, Solobacterium moorei, Parvimonas micra; or
(2) Fusobacterium nucleatum, Gemella morbillorum, Peptostreptococcus stomatis, Parvimonas micra; or
(3) Fusobacterium nucleatum, Gemella morbillorum, Peptostreptococcus stomatis, Clostridium symbiosum; or
(4) Fusobacterium nucleatum, Gemella morbillorum, Parvimonas micra, Clostridium symbiosum; or
(5) Fusobacterium nucleatum, Solobacterium moorei, Peptostreptococcus stomatis, Parvimonas micra; or
(6) Fusobacterium nucleatum, Peptostreptococcus stomatis, Parvimonas micra, Clostridium symbiosum; or
(7) Gemella morbillorum, Solobacterium moorei, Parvimonas micra, Clostridium symbiosum; or
(8) Gemella morbillorum, Peptostreptococcus stomatis, Parvimonas micra, Clostridium symbiosum; or
(9) Solobacterium moorei, Peptostreptococcus stomatis, Parvimonas micra, Clostridium symbiosum.

In some embodiments, the method comprises measuring levels of at least five bacterial markers selected from the group disclosed above. In some embodiments, the method comprises measuring levels of at least five bacterial markers selected from the group consisting of Fusobacterium nucleatum, Peptostreptococcus stomatis, Parvimonas micra, Gemella morbillorum, Solobacterium moorei, and Clostridium symbiosum. In some embodiments, the method comprises measuring levels of the following five or six bacterial markers:
(1) Fusobacterium nucleatum, Gemella morbillorum, Solobacterium moorei, Peptostreptococcus stomatis, and Parvimonas micra; or
(2) Fusobacterium nucleatum, Gemella morbillorum, Solobacterium moorei, Parvimonas micra and Clostridium symbiosum; or
(3) Fusobacterium nucleatum, Gemella morbillorum, Peptostreptococcus stomatis, Parvimonas micra and Clostridium symbiosum; or
(4) Fusobacterium nucleatum, Solobacterium moorei, Peptostreptococcus stomatis, Parvimonas micra and Clostridium symbiosum; or
(5) Gemella morbillorum, Solobacterium moorei, Peptostreptococcus stomatis, Parvimonas micra and Clostridium symbiosum; or
(6) Fusobacterium nucleatum, Gemella morbillorum, Solobacterium moorei, Peptostreptococcus stomatis, Parvimonas micra and Clostridium symbiosum.

In another aspect, the present disclosure provides a kit of diagnosing colorectal 1cancer or advanced colorectal adenoma, comprising primers for detecting in a feces sample levels of at least two bacterial markers selected from the group consisting of Fusobacterium nucleatum, Peptostreptococcus stomatis, Parvimonas micra, Gemella morbillorum, Solobacterium moorei, Porphyromonas asaccharolytica, Peptostreptococcus anaerobius, Hungatella hathewayi, Streptococcus gallolyticus, Clostridium symbiosum, Prevotella copri, Prevotella nigrescens, Bacteroides clams, genotoxic pks+Escherichia coli and gene bftP from Bacteroides fragilis

In some embodiments, the primers are capable of detecting the levels of at least three, four, five or six bacterial markers selected from the group.

In yet another aspect, the present disclosure provides a method for treating colorectal cancer or advanced colorectal adenoma in a subject, the method comprising: administering to the subject a therapeutically effective amount of a drug useful for treating colorectal cancer or advanced colorectal adenoma, wherein the subject has been determined to have colorectal cancer or advanced colorectal adenoma by the method disclosed above.

In another aspect, the present disclosure provides an agent for use in manufacturing a kit of diagnosing colorectal cancer or advanced colorectal adenoma, said agent is capable of measuring in a feces sample levels of at least two, three, four, five or six bacterial markers selected from the group consisting of Fusobacterium nucleatum, Peptostreptococcus stomatis, Parvimonas micro, Gemella morbillorum, Solobacterium moorei, Porphyromonas asaccharolytica, Peptostreptococcus anaerobius, Hungatella hathewayi, Streptococcus gallolyticus, Clostridium symbiosum, Prevotella copri, Prevotella nigrescens, Bacteroides clams, genotoxic pks+Escherichia coli and gene bftP from Bacteroides fragilis.

In yet another aspect, the present disclosure provides a computer-implemented method for identifying a discriminative region within a group of sequences. In some embodiments, the method comprises:
obtaining a plurality of sequences comprising
   a group of target sequences for identifying a discriminative region within the group, and
   a group of background sequences;
decomposing each sequence within the group of target sequences into overlapping kmers, wherein each kmer has a length of 4 to 31;
identifying a pair of kmers, wherein
   the pair of kmers occurs at most once in each sequence within the group of target sequences,
   the pair of kmers has a distance ranging from 20 to 1000,
   the pair of kmers are not identical, and
   the pair of kmers occur more than a threshold number of the target sequences;
retrieving all regions flanged by the pair of kmers in the target sequences;
aligning the regions retrieved to determine that the regions are conserved;
generating a consensus sequence based on the regions retrieved;
determining that the consensus sequence does not occur in the group of background sequences; and
retaining the consensus sequence as a discriminative region for the group of target sequences.

In some embodiments, the plurality sequences are polynucleotide sequences or polypeptide sequences. In some embodiments, the plurality of polynucleotide sequences are DNA or RNA sequences. In some embodiments, the plurality of polynucleotide sequences are genomic sequences. In some embodiments, the group of target polynucleotide sequences are genomic sequences of a viral species, including HIV, HCV, and Covid-19. In some embodiments, the group of target polynucleotide sequences are genomic sequences of a bacterial species. In some embodiments, the bacterial species is a gut microbial species.

In some embodiments, the method further comprises designing a pair of primers for amplifying the discriminative region.

In some embodiments, the method further comprises filtering the kmers before the step of identifying the pair of kmers according to a criterion selected from: (i) the kmer occurs less than or more than a threshold percentage of the target sequences; (ii) the kmer has a homopolymer, dimer or trimer of more than a threshold; or (iii) the kmer has a GC content more than or less than a threshold.

In some embodiments, the regions retrieved are aligned via BLAST, BWA, or BOWTIE. In some embodiments, an alignment software, including BLAST, BWA, BOWTIE, is used to determine that the consensus sequence does not occur in the group of background sequences.

In yet another aspect, the present disclosure provides A non-transitory computer readable medium having instructions stored thereon, the instructions, when executed by a processor, cause the processor to perform the method disclosed herein.

In yet another aspect, the present disclosure provides A bacterial marker set for use in diagnosing colorectal cancer or advanced colorectal adenoma comprising at least two sequences selected from the group consisting of SEQ ID NOs: 4, 8, 12, 16, 20, 24, 28, 32, 36, 40, 44, 48, 52, 56, 60.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the schematic of the method for identifying discriminative regions for target groups of genomic sequences. Each genomic sequence is represented by a line. For example, Sequencel is represented by a line, where the solid regions represent known sequences, whereas dotted lines represent the gaps. Each gap may represent unknown information or chromosomal breaks. All genomic sequences belonging to the same group are labeled by a group number, e.g., Groupl. R denotes a list of sequences that have no group information. The number of groups can be 1 or more and R can be empty.

FIG. 2 shows the ddPCR results of using the primers for Fusobacterium nucleatum (FN), Solobacterium moorei (SM) and Gemella morbillorum (GM) to classify the healthy, advanced colorectal adenoma and CRC group.

FIG. 3 shows that the abundance of 6 bacterial markers is significantly higher in colorectal cancer samples. pep_sto: Peptostreptococcus stomatis, par_micra: Parvimonas micra, clo_sym: Clostridium symbiosum, FN: Fusobacterium nucleatum, SM: Solobacterium moorei, GM: Gemella morbillorum. Polyp: intestinal polyp; CON: control samples with no colorectal cancer or polyp as detected by colonoscopy; NAN: gastric cancer or gastritis; PE: physical examination.

FIG. 4 shows that certain combinations of two bacterial markers demonstrated significantly better results in detecting colorectal cancer or advanced colorectal adenoma as compared to single bacterial markers. pep_sto: Peptostreptococcus stomatis, par_micra: Parvimonas micra, FN: Fusobacterium nucleatum, clo_sm: Clostridium symbiosum, SM: Solobacterium moorei, GM: Gemella morbillorum. P-value generated using Delong's test: pep_sto & par_micra vs. pep_sto: 0.0289; pep_sto & par_micra vs. par_micra: 0.0182. pep_sto & FN vs. pep_sto: 0.0481; pep_sto & FN vs. FN: 0.00552. pep_sto & GM vs. pep_sto: 0.166; pep_sto & GM vs. GM: 0.334. par_micra & FN vs. par_micra: 0.226; par_micra & FN vs. FN: 0.0125. par_micra & GM vs. par_micra: 0.0171; par_micra & GM vs. GM: 0.0829. FN & GM vs. FN: 0.0239; FN & GM vs. GM: 0.157.

FIG. 5 shows that certain combinations of three bacterial markers demonstrated significantly better results in detecting colorectal cancer or advanced colorectal adenoma as compared to single bacterial markers. P-value generated using Delong's test: pep_sto & par_micra & clo_sym vs. pep_sto: 0.0583; pep_sto & par_micra & clo_sym vs. par_micra: 0.0503; pep_sto & par_micra & clo_sym vs. clo_sym: 1.79e-11. pep_sto & par_micra & FN vs. pep_sto: 0.0433; pep_sto & par_micra & FN vs. par_micra: 0.0457; pep_sto & par_micra & FN vs. FN: 0.00202; pep_sto & par_micra & SM vs. pep_sto: 0.0656; pep_sto & par_micra & SM vs. par_micra: 0.0444; pep_sto & par_micra & SM vs. SM: 1.43e-09. pep_sto & par_micra & GM vs. pep_sto: 0.0325; pep_sto & par_micra & GM vs. par_micra: 0.0528; pep_sto & par_micra & GM vs. GM: 0.0558. pep_sto & clo_sym & FN vs. pep_sto: 0.439; pep_sto & clo_sym & FN vs. clo_sym: 9.95e-10; pep_sto & clo_sym & FN vs. FN: 0.0358; pep_sto & clo_sym & GM vs. pep_sto: 0.0988; pep_sto & clo_sym & GM vs. clo_sym: 1.01e-08; pep_sto & clo_sym & GM vs. GM: 0.246. pep_sto & SM & GM vs. pep_sto: 0.0238; pep_sto & SM & GM vs. SM: 1.37e-06; pep_sto & SM & GM vs. GM: 0.0458. par_micra & sym & FN vs. par_micra: 0.119; par_micra & clo_sym & FN vs. clo_sym: 1.1e-10; par_micra & clo_sym & FN vs. FN: 0.00813. par_micra & clo_sym & SM vs. par micra: 0.0315; par_micra & clo_sym & SM vs. clo_sym: 5.43e-09; par_micra & clo_sym & SM vs. SM: 4.47e-08. par micra & clo_sym & GM vs. par micra: 0.0319; par micra & clo_sym & GM vs. clo_sym: 1.75e-09; par micra & clo_sym & GM vs. GM: 0.152. par micra & FN & SM vs. par micra: 0.0649; par micra & FN & SM vs. FN: 0.00677; par micra & FN & SM vs. SM: 7.4e-09. par micra & FN & GM vs. par_micra: 0.0661; par micra & FN & GM vs. FN: 0.00791; par micra & FN & GM vs. GM: 0.237. par micra & SM & GM vs. par micra: 0.0246; par_micra & SM & GM vs. SM: 2.12e-08; par micra & SM & GM vs. GM: 0.108.

FIG. 6 shows that certain combinations of four bacterial markers demonstrated significantly better results in detecting colorectal cancer or advanced colorectal adenoma as compared to single bacterial markers. P-value generated using Delong's test: FN & GM & SM & par_micra vs. FN:0.0019; FN & GM & SM & par_micra vs. GM:0.0322; FN & GM & SM & par_micra vs. SM:1.27e-08; FN & GM & SM & par_micra vs. par_micra: 0.0064. FN & GM & pep_sto & par_micra vs. FN: 0.00198; FN & GM & pep_sto & par_micra vs. GM: 0.0517; FN & GM & pep_sto & par_micra vs. pep_sto: 0.0305; FN & GM & pep_sto & par_micra vs. par_micra: 0.0133. FN & GM & pep_sto & clo_sym vs. FN: 0.00382; FN & GM & pep_sto & clo_sym vs. GM: 0.148; FN & GM & pep_sto & clo_sym vs. pep_sto: 0.0462; FN & GM & pep_sto & clo_sym vs. clo_sym: 4.64e-10. FN & GM & par_micra & clo_sym vs. FN: 0.0023; FN & GM & par_micra & clo_sym vs. GM: 0.12; FN & GM & par_micra & clo_sym vs. par_micra: 0.0245; FN & GM & par_micra & clo_sym vs. clo_sym: 5.93e-10. FN & SM & pep_sto & par_micra vs. FN: 0.0018; FN & SM & pep_sto & par_micra vs. SM: 9.51e-10; FN & SM & pep_sto & par_micra vs. pep_sto: 0.0506; FN & SM & pep_sto & par micra vs. par_micra: 0.0164. FN & pep_sto & par_micar & clo_sym vs. FN: 0.000687; FN & pep_sto & par_micar & clo_sym vs. pep_sto: 0.0317; FN & pep_sto & par_micar & clo_sym vs. par_micra: 0.017; FN & pep_sto & par_micar & clo_sym vs. clo_sym: 1.34e-12. GM & SM & pep_sto & par_micra vs. GM:0.0364; GM & SM & pep_sto & par_micra vs. SM: 2.62e-07; GM & SM & pep_sto & par_micra vs. pep_sto: 0.0201; GM & SM & pep_sto & par_micra vs. par_micra: 0.0344. GM & pep_sto & par_micra & clo_sym vs. GM: 0.0352; GM & pep_sto & par micra & clo_sym vs. pep_sto: 0.0115; GM & pep_sto & par micra & clo_sym vs. par_micra: 0.00884; GM & pep_sto & par micra & clo_sym vs. clo_sym: 1.03e-10. SM & pep_sto & par_micra & clo_sym vs. SM: 1.16e-07; SM & pep_sto & par_micra & clo_sym vs. pep_sto: 0.13; SM & pep_sto & par_micra & clo_sym vs. par_micra: 0.0579; SM & pep_sto & par_micra & clo_sym vs. clo_sym: 6.43e-09.

FIGS. 7A-7E show that certain combination of five bacterial markers demonstrated significantly better results in detecting colorectal cancer or advanced colorectal adenoma as compared to single bacterial markers. FIG. 7A: combination of FN & GM & SM & pep_sto & par_micra; P-value generated using Delong's test: Five markers vs. FN: 0.00168; Five markers vs. GM: 0.0403; Five markers vs. SM: 2.29e-08; Five markers vs. pep_sto: 0.0258; Five markers vs. par_micra: 0.0073. FIG. 7B: combination of FN & GM & SM & par_micra & clo_sym; P-value generated using Delong's test: Five markers vs. FN: 0.00181; Five markers vs. GM: 0.0933; Five markers vs. SM: 2.44e-08; Five markers vs. par_micra: 0.0203; Five markers vs. clo_sym: 4.7e-10. FIG. 7C: combination of FN & GM & pep_sto & par_micra & clo_sym; P-value generated using Delong's test: Five markers vs. FN: 0.00253; Five markers vs. GM: 0.145; Five markers vs. pep_sto: 0.0722; Five markers vs. par_micra: 0.0281; Five markers vs. clo_sym: 8.1e-10. FIG. 7D: combination of FN & SM & pep_sto & par_micra & clo_sym; P-value generated using Delong's test: Five markers vs. FN: 0.00201; Five markers vs. SM: 5.62e-09; Five markers vs. pep_sto: 0.0723; Five markers vs. par_micra: 0.0134; Five markers vs. clo_sym: 7.14e-10. FIG. 7E: combination of GM & SM & pep_sto & par_micra & clo_sym; P-value generated using Delong's test: Five markers vs. GM: 0.0555; Five markers vs. SM: 1.54e-08; Five markers vs. pep_sto: 0.0165; Five markers vs. par_micra: 0.013; Five markers vs. clo_sym: 3.0778e-10

FIG. 8 shows that certain combination of six bacterial markers (FN & GM & SM & pep_sto & par_micra) demonstrated significantly better results in detecting colorectal cancer or advanced colorectal adenoma as compared to single bacterial markers. P-value generated using Delong's test: Six markers vs. FN: 0.000859; Six markers vs. GM: 0.0561; Six markers vs. SM: 1.77e-08; Six markers vs. pep_sto: 0.0214; Six markers vs. par_micra: 0.0125; Six markers vs. clo_sym: 0.0125.

FIGS. 9A and 9B shows that the combination of bacterial markers and FIT (fecal immunochemical test) resulted in higher sensitivity as compared to FIT. FIG. 6A shows the ROC curves of diagnosing colorectal cancer based on FIT. FIG. 6B shows the ROC curves of diagnosing colorectal cancer based on the combination of FIT and bacterial markers.

### DETAILED DESCRIPTION OF THE DISCLOSURE

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Definitions

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a”, “an” and “the” include plural references unless the context clearly dictates otherwise.

As used herein, the term “administering” means providing a pharmaceutical agent or composition to a subject, and includes, but is not limited to, administering by a medical professional and self-administering.

The term “amount” or “level” generally refers to the quantity of a substance of interest. In the context of gut microbe, a level of the gut microbe refers to the representation of a given phylum, order, family, genera or species of microbe present in a sample, e.g., a sample from the gastrointestinal tract of a subject. In the context of a polynucleotide or polypeptide, the term “level” refers to the quantity of the polynucleotide of interest or the polypeptide of interest present in a sample. Such quantity may be expressed in the absolute terms, i.e., the total quantity of the polynucleotide or polypeptide in the sample, or in the relative terms, i.e., the concentration of the polynucleotide or polypeptide in the sample.

As used herein, the term “cancer” refers to any diseases involving an abnormal cell growth and include all stages and all forms of the disease that affects any tissue, organ or cell in the body. The term includes all known cancers and neoplastic conditions, whether characterized as malignant, benign, soft tissue, or solid, and cancers of all stages and grades including pre- and post-metastatic cancers. In general, cancers can be categorized according to the tissue or organ from which the cancer is located or originated and morphology of cancerous tissues and cells. As used herein, cancer types include, without limitation, acute lymphoblastic leukemia (ALL), acute myeloid leukemia, adrenocortical carcinoma, anal cancer, astrocytoma, childhood cerebellar or cerebral, basal-cell carcinoma, bile duct cancer, bladder cancer, bone tumor, brain cancer, cerebellar astrocytoma, cerebral astrocytoma/malignant glioma, ependymoma, medulloblastoma, supratentorial primitive neuroectodermal tumors, visual pathway and hypothalamic glioma, breast cancer, Burkitt's lymphoma, cervical cancer, chronic lymphocytic leukemia, chronic myelogenous leukemia, colorectal cancer, emphysema, endometrial cancer, ependymoma, esophageal cancer, Ewing's sarcoma, retinoblastoma, gastric (stomach) cancer, glioma, head and neck cancer, heart cancer, Hodgkin lymphoma, islet cell carcinoma (endocrine pancreas), Kaposi sarcoma, kidney cancer (renal cell cancer), laryngeal cancer, leukemia, liver cancer, lung cancer, neuroblastoma, non-Hodgkin lymphoma, ovarian cancer, pancreatic cancer, pharyngeal cancer, prostate cancer, rectal cancer, renal cell carcinoma (kidney cancer), retinoblastoma, Ewing family of tumors, skin cancer, stomach cancer, testicular cancer, throat cancer, thyroid cancer, vaginal cancer. As used herein, the term “advanced colorectal adenoma” refers to an adenoma with significant villous features (>25%), size of 1.0 cm or more, high-grade dysplasia, or early invasive cancer.

It is noted that in this disclosure, terms such as “comprises”, “comprised”, “comprising”, “contains”, “containing” and the like have the meaning attributed in United States Patent law; they are inclusive or open-ended and do not exclude additional, un-recited elements or method steps. Terms such as “consisting essentially of” and “consists essentially of” have the meaning attributed in United States Patent law; they allow for the inclusion of additional ingredients or steps that do not materially affect the basic and novel characteristics of the claimed disclosure. The terms “consists of” and “consisting of” have the meaning ascribed to them in United States Patent law; namely that these terms are close ended.

The term “complementarity” refers to the ability of a nucleic acid to form hydrogen bond(s) with another nucleic acid sequence by either traditional Watson-Crick or other non- traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%>, 70%>, 80%>, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

The terms “determining,” “assessing,” “assaying,” “measuring” and “detecting” can be used interchangeably and refer to both quantitative and semi-quantitative determinations. Where either a quantitative and semi-quantitative determination is intended, the phrase “determining a level” of a polynucleotide or polypeptide of interest or “detecting” a polynucleotide or polypeptide of interest can be used.

The term “hybridizing” refers to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences in a mixed population (e.g., a cell lysate or DNA preparation from a tissue biopsy). A “stringent hybridization” and “stringent hybridization wash conditions” in the context of nucleic acid hybridization (e.g., as in array, microarray, Southern or northern hybridizations) are sequence dependent, and are different under different environmental parameters. An extensive guide to the hybridization of nucleic acids is found in, e.g., Tijssen Laboratory Techniques in Biochemistry and Molecular Bio logy-Hybridization with Nucleic Acid Probes part I, Ch. 2, “Overview of principles of hybridization and the strategy of nucleic acid probe assays,” (1993) Elsevier, N.Y. Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook and Russell Molecular Cloning: A Laboratory Manual (3rd ed.) Vol. 1-3 (2001) Cold Spring Harbor Laboratory, Cold Spring Harbor Press, N.Y.). An example of highly stringent wash conditions is 0.15 M NaCl at 72° C. for about 15 minutes. An example of stringent wash conditions is a 0.2× SSC wash at 65° C. for 15 minutes. Often, a high stringency wash is preceded by a low stringency wash to remove background probe signal. An example medium stringency wash for a duplex of, e.g., more than 100 nucleotides, is 1× SSC at 45° C. for 15 minutes. An example of a low stringency wash for a duplex of, e.g., more than 100 nucleotides, is 4× SSC to 6× SSC at 40° C. for 15 minutes.

The term “nucleic acid” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, shRNA, single-stranded short or long RNAs, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

In general, a “protein” is a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

As used herein, the term “subject” refers to a human or any non-human animal (e.g., mouse, rat, rabbit, dog, cat, cattle, swine, sheep, horse or primate). A human includes pre and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

As used herein, the term “therapeutically effective amount” means the amount of agent that is sufficient to prevent, treat, reduce and/or ameliorate the symptoms and/or underlying causes of any disorder or disease, or the amount of an agent sufficient to produce a desired effect on a cell. In one embodiment, a “therapeutically effective amount” is an amount sufficient to reduce or eliminate a symptom of a disease. In another embodiment, a therapeutically effective amount is an amount sufficient to overcome the disease itself

The term “treatment,” “treat,” or “treating” refers to a method of reducing the effects of a cancer (e.g., breast cancer, lung cancer, ovarian cancer or the like) or symptom of cancer. Thus, in the disclosed method, treatment can refer to a 10%, 20%, 30%, 40%, 50%, 60%, 70%), 80%), 90%), or 100% reduction in the severity of a cancer or symptom of the cancer. For example, a method of treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100% or any percent reduction between 10 and 100% as compared to native or control levels. It is understood that treatment does not necessarily refer to a cure or complete ablation of the disease, condition, or symptoms of the disease or condition.

Gut Microbial Markers

The gut microbiota (formerly called gut flora or microflora) designates the population of microorganisms living in the intestine of any organism belonging to the animal kingdom (human, animal, insect, etc.). While each individual has a unique microbiota composition (60 to 80 bacterial species are shared by more than 50% of a sampled population on a total of 400-500 different bacterial species/individual), it always fulfils similar main physiological functions and has a direct impact on the individual's health: it contributes to the digestion of certain foods that the stomach and small intestine are not able to digest (mainly non-digestible fibers); it contributes to the production of some vitamins (B and K); it protects against aggressions from other microorganisms, maintaining the integrity of the intestinal mucosa; it plays an important role in the development of a proper immune system. A healthy, diverse and balanced gut microbiota is key to ensuring proper intestinal functioning.

Taking into account the major role gut microbiota plays in the normal functioning of the body and the different functions it accomplishes, it is nowadays considered as an “organ”. However, it is an “acquired” organ, as babies are born sterile; that is, intestine colonization starts right after birth and evolves afterwards.

The development of gut microbiota starts at birth. Sterile inside the uterus, the newborn's digestive tract is quickly colonized by microorganisms from the mother (vaginal, skin, breast, etc.), the environment in which the delivery takes place, the air, etc. From the third day, the composition of the intestinal microbiota is directly dependent on how the infant is fed: breastfed babies' gut microbiota, for example, is mainly dominated by Bifidobacteria, compared to babies nourished with infant formulas.

The composition of the gut microbiota evolves throughout the entire life, from birth to old age, and is the result of different environmental influences. Gut microbiota's balance can be affected during the ageing process and, consequently, the elderly have substantially different microbiota than younger adults.

While the general composition of the dominant intestinal microbiota is similar in most healthy people (4 main phyla, i.e., Finnicutes, Bacteroidetes, Actinobacteria and Proteobacteria), composition at a species level is highly personalized and largely determined by the individuals' genetic, environment and diet. The composition of gut microbiota may become accustomed to dietary components, either temporarily or permanently. Japanese people, for example, can digest seaweeds (part of their daily diet) thanks to specific enzymes that their microbiota has acquired from marine bacteria.

In recent years, the composition of gut microbiota has been associated cancers such as colorectal cancer, gastric cancer, hepatocellular carcinoma, esophageal cancer, breast cancer and lung cancer. The methods and compositions described herein are based, in part, on the discovery of certain gut microbial markers whose levels are correlated with a colorectal cancer (CRC) or advanced colorectal adenoma. In some embodiments, the gut microbial markers are selected from the group consisting of Fusobacterium nucleatum, Peptostreptococcus stomatis, Parvimonas micro, Gemella morbillorum, Solobacterium moorei, Porphyromonas asaccharolytica, Peptostreptococcus anaerobius, Hungatella hathewayi, Streptococcus gallolyticus, Clostridium symbiosum, Prevotella copri, Prevotella nigrescens, Bacteroides clams, genotoxic pks+Escherichia colt and gene bftP from Bacteroides fragilis.

The inventors of the present disclosure found that using a combination of the gut microbial markers disclosed herein can diagnose CRC with high specificity and sensitivity. In some embodiments, the method comprises measuring levels of at least two, three, four, five, six, seven, eight, nine or ten bacterial markers selected from the group described above.

In addition, the inventors of the present disclosure found that the levels of the gut microbial markers disclosed herein can be measured by detecting nucleotide sequences specific to the gut microbes. In some embodiments, measuring the levels of the bacterial markers comprising detecting at least a sequence selected from SEQ ID NOs: 4, 8, 12, 16, 20, 24, 28, 32, 36, 40, 44, 48, 52, 56, 60.

The nucleotide sequences specific to the gut microbial biomarkers can be identified using any methods known in the art. In some embodiments, the specific sequences (discriminative sequence or region) are identified using a kmer based method to find kmers that exist specific to the in-group species, and not exist in the out-group species. The in-group species were split using kmer for the whole genomes. Each kmer is aligned to the out-group genomes. If the kmer did not existed in the out-group genomes, the kmer was retain. Otherwise, the kmer was removed from the candidate k-mer groups.

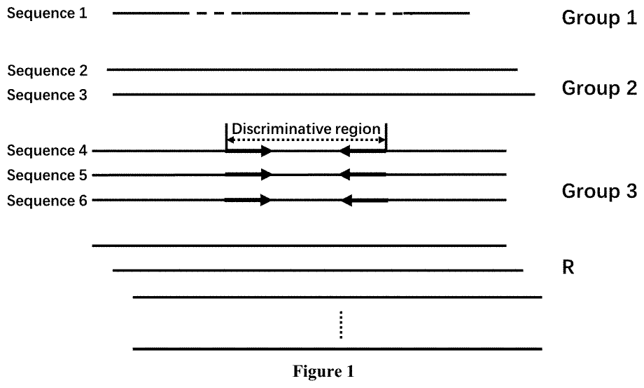

A discriminative region of a group is defined as a region that is conserved within all sequences within this group but is not-conserved outside the group. “Conservation” is measured by sequence similarity, such as the Needleman—Wunsch alignment algorithm. In some embodiments, the conservation score is defined as the ratio of number of total matched bases and alignment length by the alignment algorithm. The conservation score is typically >=0.7. In an example ass illustrated in FIG. 1, the region in between forward and reverse red arrows in Group3 are conserved regions, where the forward or reverse arrows are also conserved such that PCR primers can be designed to amplify this region among each sequence within the group or a probe can be designed to pull down such a region. There could be one or more conserved regions for each group.

In some embodiments, the kmer based method comprises:

obtaining a plurality of polynucleotide sequences comprising
a group of target polynucleotide sequences for identifying a discriminative region within the group, and
a group of background polynucleotide sequences;

decomposing each sequence within the group of target polynucleotide sequences into overlapping kmers, wherein each kmer has a length of 4 to 31;

identifying a pair of kmers, wherein
the pair of kmers occurs at most once in each sequence within the group of target polynucleotide sequences,
the pair of kmers has a distance ranging from 20 to 1000,
the pair of kmers are not identical, and
the pair of kmers occur more than a threshold number of the target polynucleotide sequences;
retrieving all regions flanged by the pair of kmers in the target polynucleotide sequences;
aligning the regions retrieved to determine that the regions are conserved; generating a consensus sequence based on the regions retrieved;
determining that the consensus sequence does not occur in the group of background polynucleotide sequences; and
retaining the consensus sequence as a discriminative region for the group of target polynucleotide sequences.

The method disclosed herein can be applied to identify discriminative or conserved regions among bacterial genomes, viral genomes, fungi genomes. It can also be used to find the conserved regions of any gene among different species. The identified set of regions and the potential application may include amplification and quantification of target, design PCR, qPCR, ddPCR experiments for target amplification, design amplicons for target sequencing.

The method is directly applicable to identify conserved regions of a set of sequences. The direct application includes designing PCR primers for single viral species, such as HIV, HCV, Covid19, TCR, and so on. This can also be directly used for identifying probe regions for pulling down targets.

The method is applicable to identify a set of targets in a cohort of organisms. For example, in the vagina or gut microbiome environment, identify regions that specifically represent a list of target species, genus, and so on.

Methods of Measuring Level of Gut Microbial Markers

In some embodiments, the method disclosed herein comprises measuring in a feces sample isolated from the subject levels of the bacterial markers disclosed herein.

Any method known to those of ordinary skill in the art can be used to measure the level of the gut microbe in the sample of the subject. In certain embodiments, the level of the gut microbe is measured by detecting the level of microbe-specific DNA in a sample, e.g., feces sample from the gut of the subject.

In some embodiments, DNA is isolated from the feces sample. DNA can be isolated from the feces sample using a variety of methods. Standard methods for DNA extraction from tissue or cells are described in, for example, Ausubel et al., Current Protocols of Molecular Biology (1997) John Wiley & Sons, and Sambrook and Russell, Molecular Cloning: A Laboratory Manual 3rd ed (2001). Commercially available kits, e.g., QIAamp® DNA Stool Mini Kit (Qiagen) can also be used to isolate DNA from a feces sample.

In certain embodiments, the level of the gut microbial markers can be detected using amplification assay, hybridization assay or sequencing assay.

Amplification Assay

A nucleic acid amplification assay involves copying a target nucleic acid (e.g., DNA or RNA), thereby increasing the number of copies of the amplified nucleic acid sequence. Amplification may be exponential or linear. Exemplary nucleic acid amplification methods include, but are not limited to, amplification using the polymerase chain reaction (“PCR”, see U.S. Pat. Nos. 4,683,195 and 4,683,202; PCR Protocols: A Guide To Methods And Applications (Innis et al., eds, 1990)), reverse transcriptase polymerase chain reaction (RT-PCR), quantitative real-time PCR (qRT-PCR); quantitative PCR, such as TaqMan®, nested PCR, ligase chain reaction (See Abravaya, K., et al., Nucleic Acids Research, 23:675-682, (1995), branched DNA signal amplification (see, Urdea, M. S., et al., AIDS, 7 (suppl 2):S11-S14, (1993), amplifiable RNA reporters, Q-beta replication (see Lizardi et al., Biotechnology (1988) 6: 1197), transcription-based amplification (see, Kwoh et al., Proc. Natl. Acad. Sci. USA (1989) 86: 1173-1177), boomerang DNA amplification, strand displacement activation, cycling probe technology, self-sustained sequence replication (Guatelli et al., Proc. Natl. Acad. Sci. USA (1990) 87:1874-1878), rolling circle replication (U.S. Pat. No. 5,854,033), isothermal nucleic acid sequence based amplification (NASBA), and serial analysis of gene expression (SAGE).

In certain embodiments, the nucleic acid amplification assay is a PCR-based method. PCR is initiated with a pair of primers that hybridize to the target nucleic acid sequence to be amplified, followed by elongation of the primer by polymerase which synthesizes the new strand using the target nucleic acid sequence as a template and dNTPs as building blocks. Then the new strand and the target strand are denatured to allow primers to bind for the next cycle of extension and synthesis. After multiple amplification cycles, the total number of copies of the target nucleic acid sequence can increase exponentially.

In certain embodiments, intercalating agents that produce a signal when intercalated in double stranded DNA may be used. Exemplary agents include SYBR GREEN™ and SYBR GOLD™. Since these agents are not template-specific, it is assumed that the signal is generated based on template-specific amplification. This can be confirmed by monitoring signals as a function of temperature because the melting point of template sequences will generally be much higher than, for example, primer-dimers, etc.

In certain embodiments, a detectably labeled primer or a detectably labeled probe can be used, to allow detection of the mRNA (or cDNA reverse transcribed from mRNA) of the gene of interest corresponding to that primer or probe. In certain embodiments, multiple labeled primers or labeled probes with different detectable labels can be used to allow simultaneous detection of the expression of multiple genes of interest.

In some embodiments, the level of the gut microbial markers described above can be detected or measured by droplet digital PCR (ddPCR). ddPCR is a refined PCR method that can be used to directly quantify and clonally amplify nucleic acids strands. Unlike conventional PCR, which performs one reaction per well, ddPCR involves partitioning the PCR solution into tens of thousands of nan-liter sized droplets, where a separate PCR reaction takes place in each one. After multiple PCR amplification cycles, the samples are checked for fluorescence with a binary readout of “0” or “1”. The fraction of fluorescing droplets is recorded. The partitioning of the sample allows one to estimate the number of different molecules by assuming that the molecule population follows the Poisson distribution, thus accounting for the possibility of multiple target molecules inhabiting a single droplet. Using Poisson's law of small numbers, the distribution of target molecule within the sample can be accurately approximated allowing for a quantification of the target strand in the PCR product. The ddPCR increases precision through massive sample partitioning, which ensures reliable measurements in the desired DNA sequence due to reproducibility.

Hybridization Assay

Nucleic acid hybridization assays use probes to hybridize to the target nucleic acid, thereby allowing detection of the target nucleic acid. Non-limiting examples of hybridization assay include Northern blotting, Southern blotting, in situ hybridization, microarray analysis, and multiplexed hybridization-based assays.

In certain embodiments, the probes for hybridization assay are detectably labeled. In certain embodiments, the nucleic acid-based probes for hybridization assay are unlabeled. Such unlabeled probes can be immobilized on a solid support, such as a microarray, and can hybridize to the target nucleic acid molecules which are detectably labeled.

In certain embodiments, hybridization assays can be performed by isolating the nucleic acids (e.g., RNA or DNA), separating the nucleic acids (e.g., by gel electrophoresis) followed by transfer of the separated nucleic acid on suitable membrane filters (e.g., nitrocellulose filters), where the probes hybridize to the target nucleic acids and allows detection. See, for example, Molecular Cloning: A Laboratory Manual, J. Sambrook et al., eds., 2nd edition, Cold Spring Harbor Laboratory Press, 1989, Chapter 7. The hybridization of the probe and the target nucleic acid can be detected or measured by methods known in the art. For example, autoradiographic detection of hybridization can be performed by exposing hybridized filters to photographic film.

In some embodiments, hybridization assays can be performed on microarrays. Microarrays provide a method for the simultaneous measurement of the levels of large numbers of target nucleic acid molecules. The target nucleic acids can be RNA, DNA, cDNA reverse transcribed from mRNA, or chromosomal DNA. The target nucleic acids can be allowed to hybridize to a microarray comprising a substrate having multiple immobilized nucleic acid probes arrayed at a density of up to several million probes per square centimeter of the substrate surface. The RNA or DNA in the sample is hybridized to complementary probes on the array and then detected by laser scanning. Hybridization intensities for each probe on the array are determined and converted to a quantitative value representing relative levels of the RNA or DNA. See, U.S. Pat. Nos. 6,040,138, 5,800,992 and 6,020,135, 6,033,860, and 6,344,316.

Techniques for the synthesis of these arrays using mechanical synthesis methods are described in, e.g., U.S. Pat. No. 5,384,261. Although a planar array surface is often employed the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays may be packaged in such a manner as to allow for diagnostics or other manipulation of an all-inclusive device. Useful microarrays are also commercially available, for example, microarrays from Affymetrix, from Nano String Technologies, QuantiGene 2.0 Multiplex Assay from Panomi cs.

Sequencing Methods

Sequencing methods useful in the measurement of the level of the gut microbial markers involves sequencing of the nucleic acid specific to the gut microbial markers. In general, sequencing methods can be categorized to traditional or classical methods and high throughput sequencing (next generation sequencing). Traditional sequencing methods include Maxam-Gilbert sequencing (also known as chemical sequencing) and Sanger sequencing (also known as chain-termination methods).

High throughput sequencing, or next generation sequencing, by using methods distinguished from traditional methods, such as Sanger sequencing, is highly scalable and able to sequence the entire genome or transcriptome at once. High throughput sequencing involves sequencing-by-synthesis, sequencing-by-ligation, and ultra-deep sequencing (such as described in Marguiles et al., Nature 437 (7057): 376-80 (2005)). Sequence-by-synthesis involves synthesizing a complementary strand of the target nucleic acid by incorporating labeled nucleotide or nucleotide analog in a polymerase amplification. Immediately after or upon successful incorporation of a label nucleotide, a signal of the label is measured and the identity of the nucleotide is recorded. The detectable label on the incorporated nucleotide is removed before the incorporation, detection and identification steps are repeated. Examples of sequence-by-synthesis methods are known in the art, and are described for example in U.S. Pat. Nos. 7,056,676, 8,802,368 and 7,169,560, the contents of which are incorporated herein by reference. Sequencing-by-synthesis may be performed on a solid surface (or a microarray or a chip) using fold-back PCR and anchored primers. Target nucleic acid fragments can be attached to the solid surface by hybridizing to the anchored primers, and bridge amplified. This technology is used, for example, in the Illumina® sequencing platform.

Pyrosequencing involves hybridizing the target nucleic acid regions to a primer and extending the new strand by sequentially incorporating deoxynucleotide triphosphates corresponding to the bases A, C, G, and T (U) in the presence of a polymerase. Each base incorporation is accompanied by release of pyrophosphate, converted to ATP by sulfurylase, which drives synthesis of oxyluciferin and the release of visible light. Since pyrophosphate release is equimolar with the number of incorporated bases, the light given off is proportional to the number of nucleotides adding in any one step. The process is repeated until the entire sequence is determined.

Machine Learning Classification

In some embodiments, the method disclosed herein comprises classify the subject as healthy or having colorectal cancer or advanced colorectal adenoma based on the measured levels of the bacterial markers. In some embodiments, the method comprises evaluating the measured levels of the bacterial markers by a machine learning classifier, and determining that the subject is healthy or has colorectal cancer or advanced colorectal adenoma.

In statistics, classification is the problem of identifying which of a set of categories an observation (or observations) belongs to. As used herein, classification refers to the identification of the subject as being healthy or having colorectal cancer or adenoma based on the measured levels of the bacterial markers. A “classifier” refers to an algorithm that implements the classification.

Commonly used classification algorithms include linear classification (e.g., Fisher's linear discriminant, logistic regression, naive Bayes classifier, and perceptron), support vector machines (e.g., least squares support vector machines), quadratic classifiers, Kernel estimation (e.g., k-nearest neighbor), Boosting (meta-algorithm), decision trees (e.g., random forests), neural networks, and learning vector quantization.

In general, to perform a classification with a classifier, training dataset which have been labeled with predetermined categories (class) are fed to the classifier. The classifier builds a classification model based on the training dataset (i.e., predict the class). The classification model is then used to analyze the target data (e.g., measured levels of the bacterial biomarkers).

In certain embodiments, the machine learning classifier used herein is random forest or logistic regression.

Random forest is a method that operates by constructing a multitude of decision trees at training time and outputs the class that is the mode of the mode of the classes or classification or mean prediction of the individual trees. A random forest is a meta-estimator that fits a number of trees on various subsamples of data sets and then uses an average to improve the accuracy in the model's predictive nature. In general, the random forest is more accurate than the decision trees due to the reduction in the over-fitting.

Logistic regression uses one or more independent variables to determine an outcome. The outcome is measured with a dichotomous variable (i.e., it will have only two possible outcomes). The goal of logistic regression is to find a best-fitting relationship between the dependent variable and a set of independent variables. It is better than other binary classification algorithms as it quantitatively explains the factors leading to classification.

Computer-Implemented Methods, Systems and Devices

Any of the methods described herein may be totally or partially performed with a computer system including one or more processors, which can be configured to perform the steps. Thus, embodiments are directed to computer systems configured to perform the steps of any of the methods described herein, potentially with different components performing a respective step or a respective group of steps. Although presented as numbered steps, steps of methods herein can be performed at a same time or in a different order. Additionally, portions of these steps may be used with portions of other steps from other methods. Also, all or portions of a step may be optional. Any of the steps of any of the methods can be performed with modules, circuits, or other means for performing these steps.

Any of the computer systems mentioned herein may utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. The subsystems can be interconnected via a system bus. Additional subsystems include, for examples, a printer, keyboard, storage device(s), monitor, which is coupled to display adapter, and others. Peripherals and input/output (I/O) devices, which couple to I/O controller, can be connected to the computer system by any number of means known in the art, such as serial port. For example, serial port or external interface (e.g. Ethernet, Wi-Fi, etc.) can be used to connect computer system to a wide area network such as the Internet, a mouse input device, or a scanner. The interconnection via system bus allows the central processor to communicate with each subsystem and to control the execution of instructions from system memory or the storage device(s) (e.g., a fixed disk, such as a hard drive or optical disk), as well as the exchange of information between subsystems. The system memory and/or the storage device(s) may embody a computer readable medium. Any of the data mentioned herein can be output from one component to another component and can be output to the user.

A computer system can include a plurality of the same components or subsystems, e.g., connected together by external interface or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

It should be understood that any of the embodiments of the present disclosure can be implemented in the form of control logic using hardware (e.g., an application specific integrated circuit or field programmable gate array) and/or using computer software with a generally programmable processor in a modular or integrated manner. As used herein, a processor includes a multi-core processor on a same integrated chip, or multiple processing units on a single circuit board or networked. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C++ or Perl using, for example, conventional or object-oriented techniques. The software code may be stored as a series of instructions or commands on a computer readable medium for storage and/or transmission, suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Kits and Microarrays

In another aspect, the present disclosure provides kits for use in the methods described above. The kits may comprise any or all of the reagents to perform the methods described herein. In certain embodiments, the kit comprises primers for detecting the nucleic acids specific to the gut microbial markers in a sample.

“Primer” as used herein refers to an oligonucleotide molecule with a length of 7-40 nucleotides, preferablyl0-38 nucleotides, preferably 15-30 nucleotides, or 15-25 nucleotides, or 17-20 nucleotides. For example, the primer can an oligonucleotide having a length of 7, 8, 9, 10, 11, 12, 13, 14, 15, 16, 17, 18, 19, 20, 21, 22, 23, 24, 25, 26, 27, 28, 29 or 30 nucleotides. Primers are used in the amplification of a DNA sequence by polymerase chain reaction (PCR) as well known in the art. For a DNA template sequence to be amplified, a pair of primers can be designed at its 5′ upstream and its 3′ downstream sequence, i.e. , 5′ primer and 3′ primer, each of which can specifically hybridize to a separate strand of the DNA double strand template. 5′ primer is complementary to the anti-sense strand of the DNA double strand template; and 3′ primer is complementary to the sense strand of the DNA template. As known in the art, the “sense strand” of a double stranded DNA template is the strand which contains the sequence identical to the mRNA sequence transcribed from the DNA template (except that “U” in RNA corresponds to “T” in the DNA) and encoding for a protein product. The complementary sequence of the sense strand is the “anti-sense strand.” In the present disclosure, all the SEQ ID NOs are sense strand DNA, and the sequences to which the SEQ ID NOs are complementary are anti-sense strand DNA.

In certain embodiments, the kit further comprises an agent for amplifying the target nucleic acid using the primers. In addition, the kits may include instructional materials containing directions (i.e., protocols) for the practice of the methods provided herein. While the instructional materials typically comprise written or printed materials they are not limited to such. Any medium capable of storing such instructions and communicating them to an end user is contemplated by this disclosure. Such media include, but are not limited to electronic storage media (e.g., magnetic discs, tapes, cartridges, chips), optical media (e.g., CD ROM), and the like. Such media may include addresses to interne sites that provide such instructional materials.

In another aspect, the present disclosure provides oligonucleotide probes for detecting the nucleic acids specific to the gut microbial markers in a sample. In certain embodiments, the probes are attached to a solid support, such as an array slide or chip, e.g., as described in Eds., Bowtell and Sambrook DNA Microarrays: A Molecular Cloning Manual (2003) Cold Spring Harbor Laboratory Press. Construction of such devices are well known in the art, for example as described in US Patents and Patent Publications U.S. Pat. No. 5,837,832; PCT application WO95/11995; U.S. Pat. No. 5,807,522; 7,157,229, 7,083,975, 6,444,175, 6,375,903, 6,315,958, 6,295,153, and 5,143,854, 2007/0037274, 2007/0140906, 2004/0126757, 2004/0110212, 2004/0110211, 2003/0143550, 2003/0003032, and 2002/0041420. Nucleic acid arrays are also reviewed in the following references: Biotechnol Annu Rev (2002) 8:85-101; Sosnowski et al. Psychiatr Genet (2002)12(4): 181-92; Heller, Annu Rev Biomed Eng (2002) 4: 129-53; Kolchinsky et al., Hum. Mutat (2002) 19(4):343-60; and McGail et al., Adv Biochem Eng Biotechnol (2002) 77:21-42.

A microarray can be composed of a large number of unique, single-stranded polynucleotides, usually either synthetic antisense polynucleotides or fragments of cDNAs, fixed to a solid support. Typical polynucleotides are preferably about 6-60 nucleotides in length, more preferably about 15-30 nucleotides in length, and most preferably about 18-25 nucleotides in length. For certain types of arrays or other detection kits/systems, it may be preferable to use oligonucleotides that are only about 7-20 nucleotides in length. In other types of arrays, such as arrays used in conjunction with chemiluminescent detection technology, preferred probe lengths can be, for example, about 15-80 nucleotides in length, preferably about 50-70 nucleotides in length, more preferably about 55-65 nucleotides in length, and most preferably about 60 nucleotides in length.

Techniques for the synthesis of these arrays using mechanical synthesis methods are described in, e.g., U.S. Pat. No. 5,384,261. Although a planar array surface is often employed the array may be fabricated on a surface of virtually any shape or even a multiplicity of surfaces. Arrays may also be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays may be packaged in such a manner as to allow for diagnostics or other manipulation of an all-inclusive device.

The probes and primers necessary for practicing the present disclosure can be synthesized and labeled using well known techniques. Oligonucleotides used as probes and primers may be chemically synthesized according to the solid phase phosphoramidite triester method first described by Beaucage and Caruthers, Tetrahedron Letts. (1981) 22: 1859-1862, using an automated synthesizer, as described in Needham-Van Devanter et al, Nucleic Acids Res. (1984) 12:6159-6168.

Methods for Treating Cancer

In yet another aspect, the present disclosure provides a method for treating colorectal cancer or advanced colorectal adenoma in a subject. In some embodiments, the method comprises administering to the subject a therapeutically effective amount of a drug useful for treating colorectal cancer or advanced colorectal adenoma, wherein the subject has been determined to have colorectal cancer or advanced colorectal adenoma by a machine learning classifier based on levels of at least two bacterial markers measured in a feces sample isolated from the subject, wherein the bacterial markers are selected from the group disclosed herein.

The drug that can be used in the method disclosed herein include, without limitation: alkylating agents or agents with an alkylating action, such as cyclophosphamide (CTX; e.g. cytoxan®), chlorambucil (CHL; e.g. leukeran®), cisplatin (CisP; e.g. platinol®) busulfan (e.g. myleran®), melphalan, carmustine (BCNU), streptozotocin, triethylenemelamine (TEM), mitomycin C, and the like; anti-metabolites, such as methotrexate (MTX), etoposide (VP16; e.g. vepesid®), 6-mercaptopurine (6MP), 6-thiocguanine (6TG), cytarabine (Ara-C), 5-fluorouracil (5-FU), capecitabine (e.g.Xeloda®), dacarbazine (DTIC), and the like; antibiotics, such as actinomycin D, doxorubicin (DXR; e.g. adriamycin®), daunorubicin (daunomycin), bleomycin, mithramycin and the like; alkaloids, such as vinca alkaloids such as vincristine (VCR), vinblastine, and the like; and other antitumor agents, such as paclitaxel (e.g. taxol®) and pactitaxel derivatives, the cytostatic agents, glucocorticoids such as dexamethasone (DEX; e.g. decadron®) and corticosteroids such as prednisone, nucleoside enzyme inhibitors such as hydroxyurea, amino acid depleting enzymes such as asparaginase, leucovorin, folinic acid, raltitrexed, and other folic acid derivatives, and similar, diverse antitumor agents. The following agents may also be used as additional agents: amifostine (e.g. ethyol®), dactinomycin, mechlorethamine (nitrogen mustard), streptozocin, cyclophosphamide, lomustine (CCNU), doxorubicin lipo (e.g. doxil®), gemcitabine (e.g. gemzar®), daunorubicin lipo (e.g. daunoxome®), procarbazine, mitomycin, docetaxel (e.g. taxotere®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon alpha, interferon beta, mitoxantrone, topotecan, leuprolide, megestrol, melphalan, mercaptopurine, plicamycin, mitotane, pegaspargase, pentostatin, pipobroman, plicamycin, teniposide, testolactone, thioguanine, thiotepa, uracil mustard, vinorelbine, and chlorambucil.

In some embodiment, the drug used in the method disclosed herein include, without limitation: Alymsys® (Bevacizumab), Avastin® (Bevacizumab), Camptosar® (Irinotecan Hydrochloride), Capecitabine, Cetuximab, Cyramza® (Ramucirumab), Eloxatin® (Oxaliplatin), Erbitux® (Cetuximab), 5-FU (Fluorouracil Injection), Fluorouracil Injection, Ipilimumab, Irinotecan Hydrochloride, Keytruda® (Pembrolizumab), Leucovorin Calcium, Lonsurf® (Trifluridine and Tipiracil Hydrochloride), Mvasi® (Bevacizumab), Opdivo® (Nivolumab), Oxaliplatin, Panitumumab, Pembrolizumab, Ramucirumab, Regorafenib, Stivarga® (Regorafenib), Trifluridine and Tipiracil Hydrochloride, Vectibix® (Panitumumab), Xeloda® (Capecitabine), Yervoy® (Ipilimumab), Zaltrap® (Ziv-Aflibercept), Zirabev® (Bevacizumab), Ziv-Aflibercept.

The drug described herein may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, the drug may be administered in conjunction with other treatments.

The following examples are provided to better illustrate the claimed disclosure and are not to be interpreted as limiting the scope of the disclosure. All specific compositions, materials, and methods described below, in whole or in part, fall within the scope of the present disclosure. These specific compositions, materials, and methods are not intended to limit the disclosure, but merely to illustrate specific embodiments falling within the scope of the disclosure. One skilled in the art may develop equivalent compositions, materials, and methods without the exercise of inventive capacity and without departing from the scope of the disclosure. It will be understood that many variations can be made in the procedures herein described while still remaining within the bounds of the present disclosure. It is the intention of the inventors that such variations are included within the scope of the disclosure.

### EXAMPLE 1

This example shows the identification of gut microbial markers for colorectal cancer.

A specific microbial database of human gut was constructed based on the NCBI RefSeq database of bacteria and literature-based database including: (a) uhgg database (Almeida A, et al., A unified catalog of 204,938 reference genomes from the human gut microbiome. Nature Biotechnology, 2021, 39(1): 105-114) and (b) GMrepo database (Wu S, et al., GMrepo: a database of curated and consistently annotated human gut metagenomes. Nucleic Acids Research, 2020, 48(D1): D545-D553). The inventors then searched a number of public studies about colorectal cancer (CRC) to locate intestinal microbial markers for CRC screening. The inventors also searched all gut microbes related to CRC in public and accessible literatures using Natural Language Processing (NLP), combined with manually check their reliability, to select (sub)species and strains occurred across at least two literatures. Altogether, the inventors identified 13 species markers, 1 strain marker and 1 gene marker as listed in Table 1.

TABLE 1
15 targeted gut microbial biomarkers

| Biomarker Type | Biomarker ID | Biomarker Name |
|---|---|---|
| species | 851 | Fusobacterium nucleatum |
| species | 341694 | Peptostreptococcus stomatis |
| species | 33033 | Parvimonas micra |
| species | 29391 | Gemella morbillorum |
| species | 102148 | Solobacterium moorei |
| species | 28123 | Porphyromonas asaccharolytica |
| species | 1261 | Peptostreptococcus anaerobius |
| species | 154046 | Hungatella hathewayi |
| species | 315405 | Streptococcus gallolyticus |
| species | 1512 | [Clostridium] symbiosum |
| species | 165179 | Prevotella copri |
| species | 28133 | Prevotella nigrescens |
| species | 626929 | Bacteroides clarus |
| Strain | pks | genotoxic pks + Escherichia coli |
| gene | bft | gene bft from Bacteroides fragilis |

Gene pks is hybrid polyketide-nonribosomal peptide synthase operon (pks, also referred to as clb) responsible for the production of the genotoxin colibactin. Gene bftP encodes metalloprotease enterotoxin.

### EXAMPLE 2

This example illustrates the identification of discriminative regions for each bacterial marker. The genomic sequences for each gut microbial biomarkers identified in Example 1 were retrieved from the RefSeq database. The sequences belonging to the same microbial marker were classified as in the same group.

The inventors first identified all potential anchoring kmers. Each sequence was first decomposed into overlapping kmers, where k was typically in the range of 4 to 31. A kmer is a string with length k. However, certain bases can be skipped, and spaced-seed kmer can be used. Each kmer and its position on the sequence was recorded. Each kmer serves as a seed that anchors conserved regions within the group and discriminative regions between the group.

Filter out any kmer if it satisfies one of the following: (i) low frequency, if the kmer occurs in less than a certain number of sequences within the group; (ii) high frequency, if the kmer is too frequently occurred, likely to be from a repeat region (iii) Low complexity, if the kmer has a homopolymer or dimer or trimer more than a set threshold; (iv) Other criteria. For example, additional criterion can be applied to filter the kmer, such as constraining the GC fraction.

The inventors then determined potential conserved regions by generating kmer pairs that anchor the region, like illustrated in Figurel Group3. Candidate kmer pairs need to satisfy length constraints, which can range from 20 bp to 1000 bp or more. Each kmer pair occur at most once in each of the sequence of group i. The two kmers in the pair are not identical to each other. Retain kmer pairs that occur more than a set number of sequences in group i. For each kmer pair identified, retrieve all regions anchored by these two kmers in all sequences of group i. The inventors call these regions amplicons of the kmer pair. Retain kmer pair if the following criteria are satisfied: (a) Number of amplicons is greater than a threshold; (b) Pairwisely, the amplicons have conservation score greater than a set threshold.

A consensus amplicon sequence was then generated for each kmer pair. Multiple sequence alignment of amplicons was applied. Dominant base was taken as consensus base for each amplicon position, ties were broken arbitrarily.

For each consensus amplicon sequence generated for group i, any sequence not in group i was checked against, which could be done by using any alignment software such as BLAST, BWA, BOWTIE, and so on. The amplicon sequence was retained as candidate region for group i if no significant hit was found.

Further, the identified amplicon sequences for each group i were used for primer pair design for downstream analysis such as PCR, qPCR, ddPCR, amplicon sequencing.

### EXAMPLE 3

This example illustrates the validation of multiplex primer sets using qPCR assay and ddPCR assay. Exemplary primer sets, probs and amplicon sequences are listed in Table 2 below.

TABLE 2
Primers, Probes and Amplicons for Detecting Bacterial Markers.

| | Forward Primer | Reverse Primer | Probe | Amplicon |
|---|---|---|---|---|
| Peptostreptococcus | SEQ ID NO: 1 | SEQ ID NO: 2 | SEQ ID NO: 3 | SEQ ID NO: 4 |
| anaerobius | | | | |
| Prevotella copri | SEQ ID NO: 5 | SEQ ID NO: 6 | SEQ ID NO: 7 | SEQ ID NO: 8 |
| Peptostreptococcus | SEQ ID NO: 9 | SEQ ID NO: 10 | SEQ ID NO: 11 | SEQ ID NO: 12 |
| stomatis | | | | |
| Polyketide | SEQ ID NO: 13 | SEQ ID NO: 14 | SEQ ID NO: 15 | SEQ ID NO: 16 |
| synthetase | | | | |
| Porphyromonas | SEQ ID NO: 17 | SEQ ID NO: 18 | SEQ ID NO: 19 | SEQ ID NO: 20 |
| asaccharolytica | | | | |
| Streptococcus | SEQ ID NO: 21 | SEQ ID NO: 22 | SEQ ID NO: 23 | SEQ ID NO: 24 |
| gallolyticus | | | | |
| Hungatella | SEQ ID NO: 25 | SEQ ID NO: 26 | SEQ ID NO: 27 | SEQ ID NO: 28 |
| hathewayi | | | | |
| Prevotella | SEQ ID NO: 29 | SEQ ID NO: 30 | SEQ ID NO: 31 | SEQ ID NO: 32 |
| nigrescens | | | | |
| Parvimonas micra | SEQ ID NO: 33 | SEQ ID NO: 34 | SEQ ID NO: 35 | SEQ ID NO: 36 |
| Enterotoxigenic | SEQ ID NO: 37 | SEQ ID NO: 38 | SEQ ID NO: 39 | SEQ ID NO: 40 |
| bacteroides fragilis | | | | |
| Bacteroides clarus | SEQ ID NO: 41 | SEQ ID NO: 42 | SEQ ID NO: 43 | SEQ ID NO: 44 |
| Clostridium | SEQ ID NO: 45 | SEQ ID NO: 46 | SEQ ID NO: 47 | SEQ ID NO: 48 |
| symbiosum | | | | |
| Fusobacterium | SEQ ID NO: 49 | SEQ ID NO: 50 | SEQ ID NO: 51 | SEQ ID NO: 52 |
| nucleatum | | | | |
| Solobacterium | SEQ ID NO: 53 | SEQ ID NO: 54 | SEQ ID NO: 55 | SEQ ID NO: 56 |
| moorei | | | | |
| Gemella | SEQ ID NO: 57 | SEQ ID NO: 58 | SEQ ID NO: 59 | SEQ ID NO: 60 |
| morbillorum | | | | |

The quantitative PCR (qPCR) reaction was performed in the ABI 7500 qPCR System (Thermo Fisher Scientific). The reaction mix (18 μl) was prepared as follows: take 2m1 centrifuge tube, for each reaction, add primer F&R (10 μM) 1.8 μL, Probe (10 μM) 0.5 μL, RNase Free Water 3.9 μL, TaqMan Fast Advanced Master Mix (Thermo Fisher Scientific) 10 μL. The reaction mix was vortexed and centrifuged for 30-40s without bubbles. The reaction plate (20 μl) was prepared as follows: added the reaction mix to 8-Tube Strips, added plasmid DNA (50 ng/μL) 2 μL, or no-template controls (NTCs) with nuclease free water 2 μL. The reaction plate was vortexed and centrifuged for 30-40s without bubble. Place the 8-Tube Strips in ABI 7500 qPCR System, and the reaction procedure was as follows: uracil-N glycosylase (UNG) incubation: 50° C., 2 min; polymerase activation: 95° C., 2 min; PCR (40 cycles): Denature 95° C., 3s; anneal/extend 60° C., 30s.

The Droplet Digital PCR (ddPCR) reaction was performed in the QX200M Droplet Digital PCR system (BIO-RAD). The reaction mix (22 μl) was prepared as follows: primer F&R (10 μM) 1.98 μL, probe (10 μM) 0.55 μL, nuclease free Water 4.29 μL, TaqMan Fast Advanced Master Mix 11 μL, sample DNA 2.2 Blow and mix the reaction solution, add 20 μl to each well of the reaction mix. The reaction plate was prepared as follows: loading a 20 μl PCR reaction into the well, then loaded 70 μl of droplet generation oil into the bottom wells of the DG8 cartridge, placed it into the QX200 droplet generator. The generated oil-water mixture was slowly extracted 40 μl to the ddPCR 96-well PCR plates, which were covered with an aluminum film and placed the PX1 PCR plate sealer, which had been heated to 180° C. The reaction plate was then loaded as follows: place the 96-Well PCR Plates in ABI 7500 qPCR System, and the reaction procedure was as follows: 95° C., 5 min; PCR (40 cycles): 95° C., 30s; 60° C., 10 min; 98° C., 10 min. After the PCR amplification, place the PCR plate was placed in the QX200 droplet reader to read the droplet.

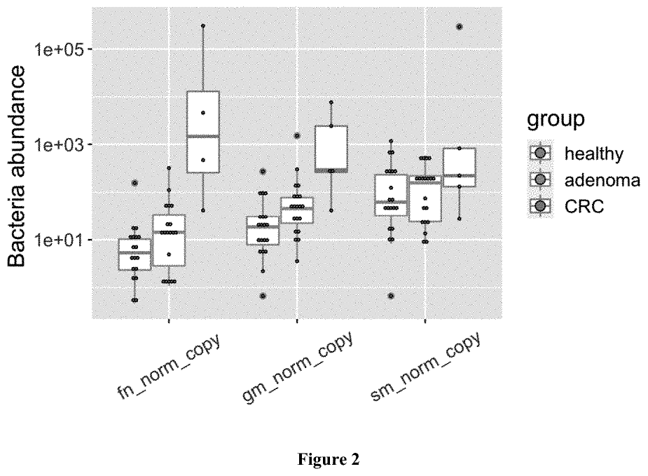

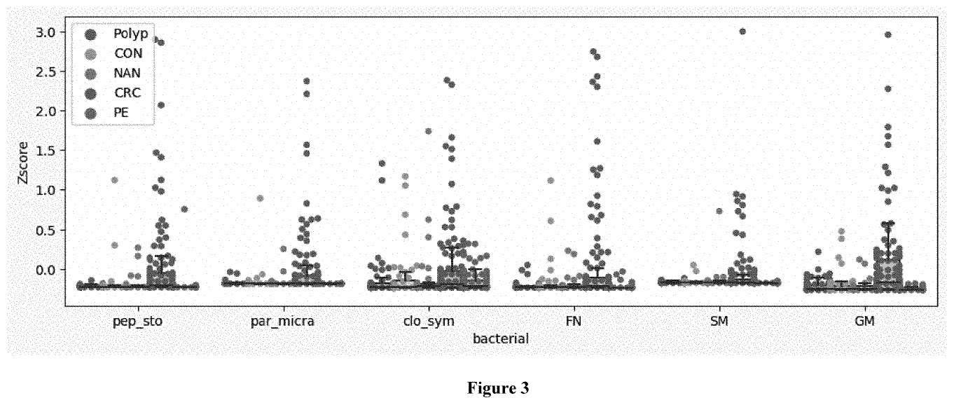

As shown in FIG. 2, the ddPCR results using the primers for Fusobacterium nucleatum (FN), Solobacterium moorei (SM) and Gemella morbillorum (GM) showed the different abundance of the bacterial in healthy, advanced colorectal adenoma and colorectal cancer groups.

### EXAMPLE 4

This example illustrates the diagnosis of colorectal cancer using a combination of multiple bacterial markers.

The inventors first measured the abundance of the bacterial markers in the feces samples from different samples including intestinal polyp (polyp), control healthy subject (CON), gastric cancer or gastritis (NAN), non-advanced adenoma (NAA), colorectal cancer (CRC), physical examination (PE).

Primers and probes specific to the bacterial markers were designed. Total DNA were extracted from feces samples and were used as template for DNA amplification using the specific primers and ddPCR, generating the copy number (abundance) of each bacterial marker in 100 ng total DNA. The results of a bacterial marker in each sample were adjusted by z-score according to the following formula:

(The abundance of a bacterial marker in a specific sample — the average abundance of the bacterial marker in all samples)/the standard deviation of the abundance of the bacterial marker in all samples

As illustrated in FIG. 3, the abundance of 6 bacterial markers Peptostreptococcus stomatis(pep_sto), Parvimonas micra (par_micra), Clostridium symbiosum (clo_sym), Fusobacterium nucleatum (FN), Solobacterium moorei (SM), Gemella morbillorum (GM) is significantly higher in colorectal cancer samples.

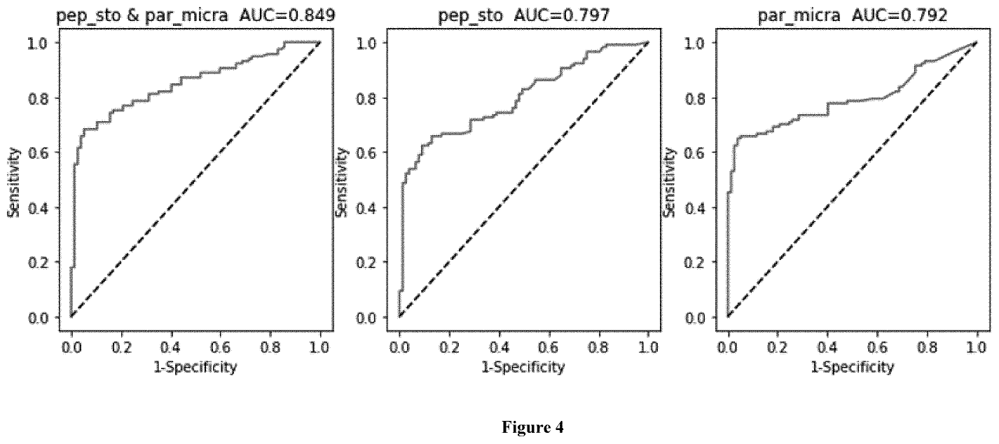

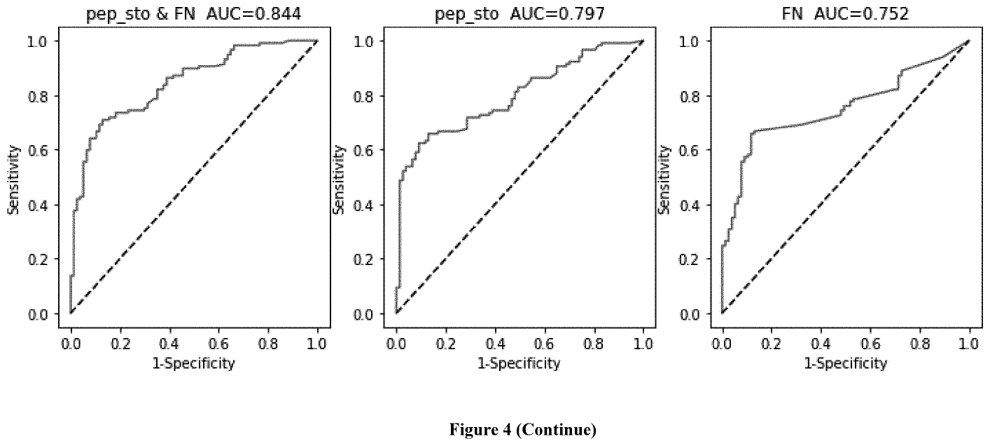

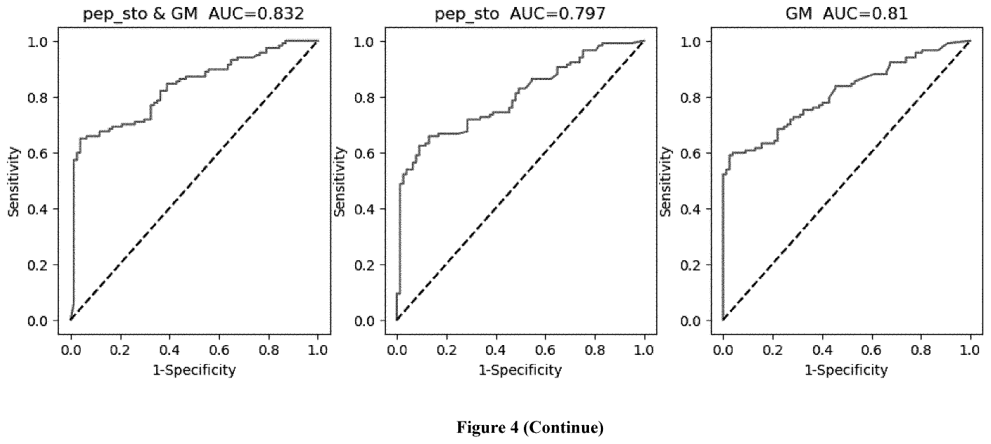

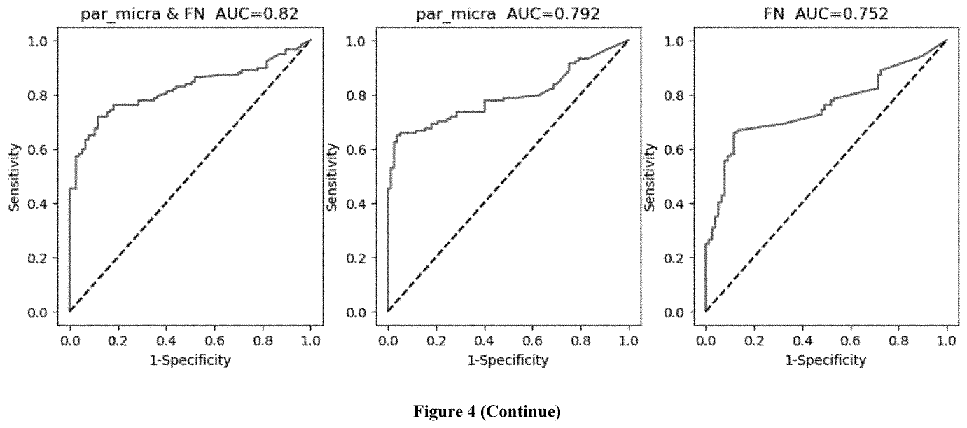

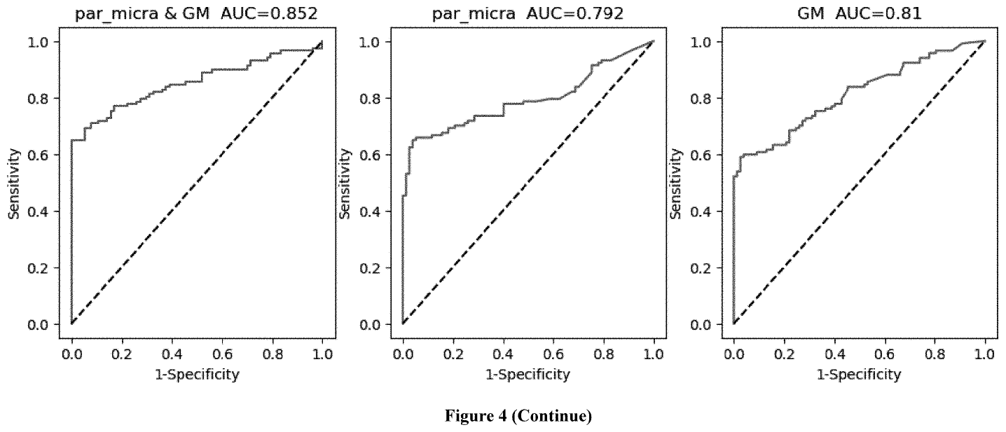

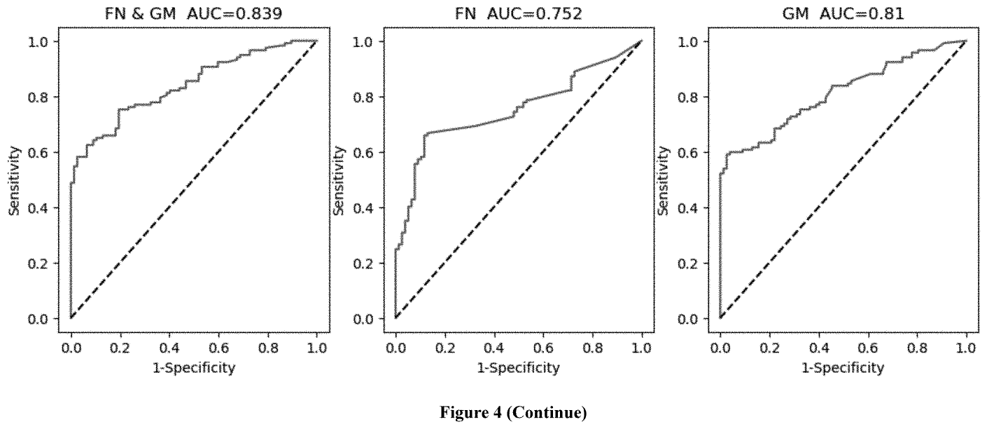

The inventors then compared the diagnosis of colorectal cancer using a single bacterial marker with using a combination of multiple bacterial markers. The analysis used 121 CRC samples and 78 PE samples. The abundance of each bacterial marker (copy number in 100 ng total DNA extracted from the fecal sample) was measured using ddPCR as described above. A logistic regression classifier was trained using 5-fold cross validation to generate hyper-parameters. The logistic regression classifier was then re-trained with the hyper-parameters and used for test prediction. To compare whether the ROC curves generated by different classifiers are significantly different, p-value was generated using Delong's test. The results (p-value as compared to single bacterial marker) of the combination of two bacterial markers are shown in FIG. 4 and Table 3 below.

TABLE 3
The comparison of diagnosis using single bacterial marker with
using two bacterial markers.

| | FN | Pep_sto | Par_micra | GM | SM | Clo_sum |
|---|---|---|---|---|---|---|
| FN | | 0.0055 | 0.0125 | 0.0239 | 0.275 | 0.797 |
| | | 0.0481 | 0.226 | 0.157 | 0.0002 | 1.55e−06 |
| Pep_sto | | | 0.0289 | 0.166 | 0.982 | 0.952 |
| | | | 0.0182 | 0.334 | 4.615 | 4.57e−08 |
| Par_micra | | | | 0.0171 | 0.492 | 0.765 |
| | | | | 0.0829 | 2.19e−07 | 3.318 |
| GM | | | | | 0.456 | 0.961 |
| | | | | | 0.0000376 | 1.05e−06 |
| SM | | | | | | 0.0043 |
| | | | | | | 8.13e−05 |
| Clo_sym | | | | | | |

As shown in FIG. 4 and Table 3, the combination, illustrated in anyone of the following groups (1)-(6), of two bacterial markers demonstrated significantly better results compared to using a single bacterial marker: (1) Peptostreptococcus stomatis and Parvimonas micra; (2) Peptostreptococcus stomatis and Fusobacterium nucleatum; (3) Peptostreptococcus stomatis and Gemella morbillorum; (4) Fusobacterium nucleatum and Parvimonas micra; (5) Gemella morbillorum and Parvimonas micra; (6) Fusobacterium nucleatum and Gemella morbillorum.

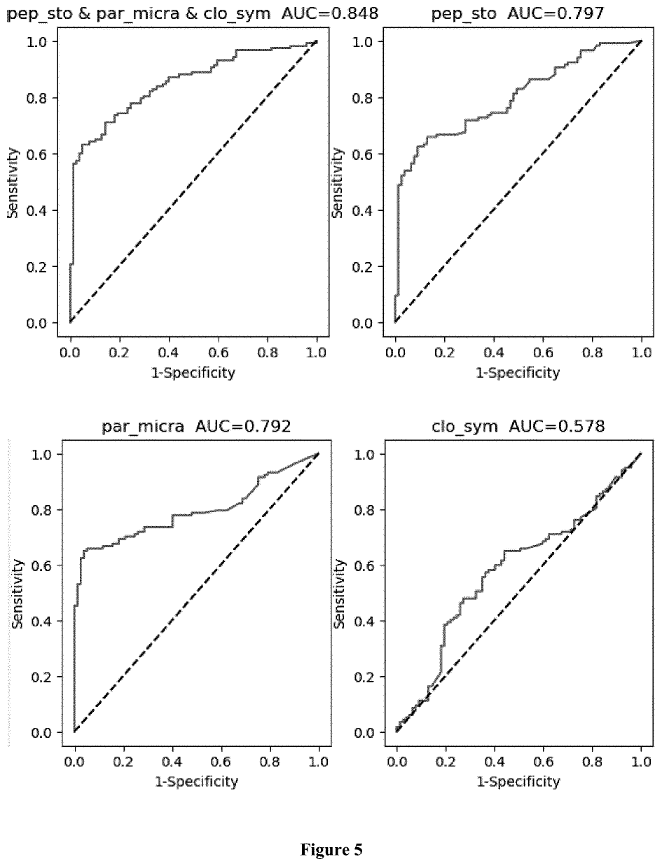

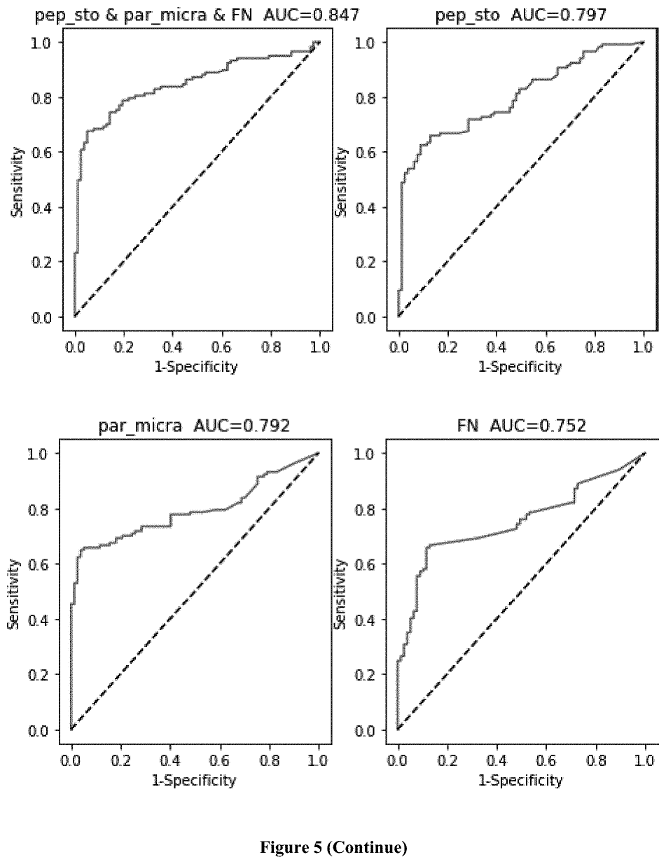

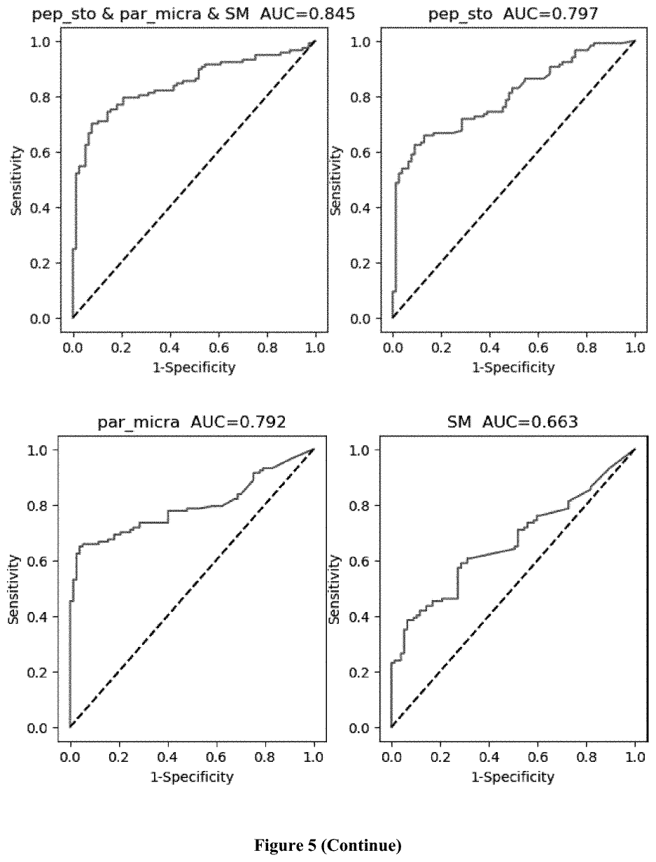

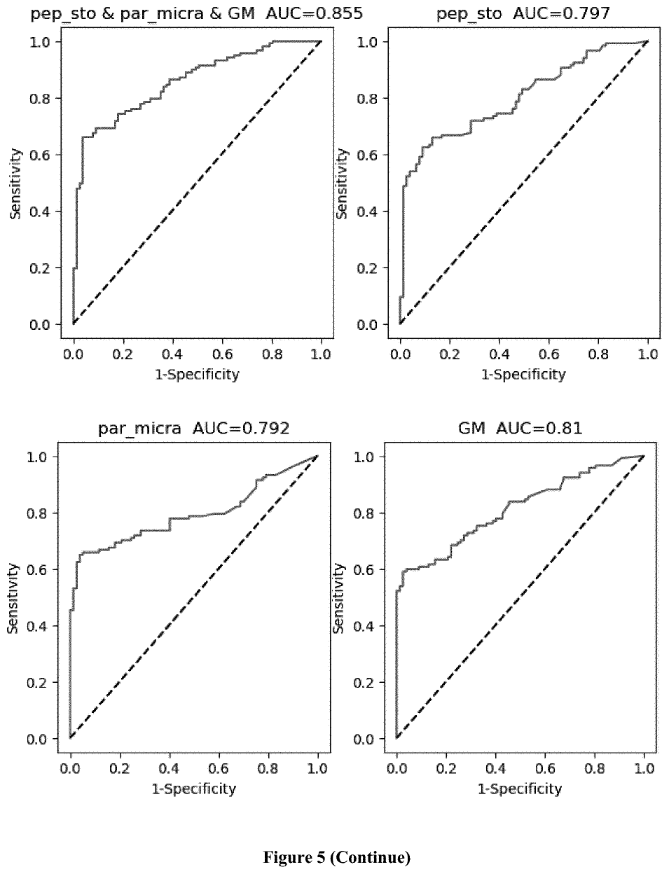

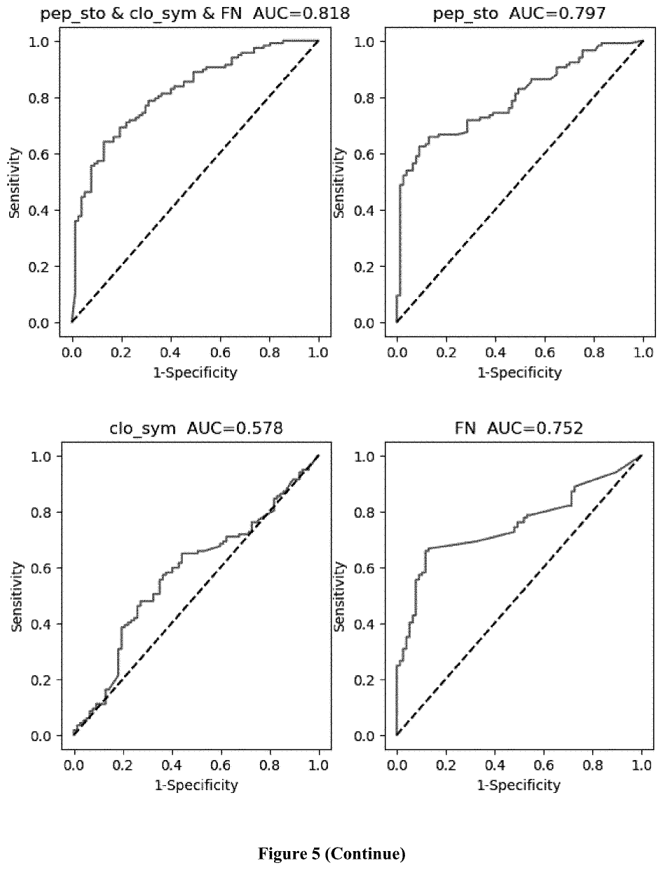

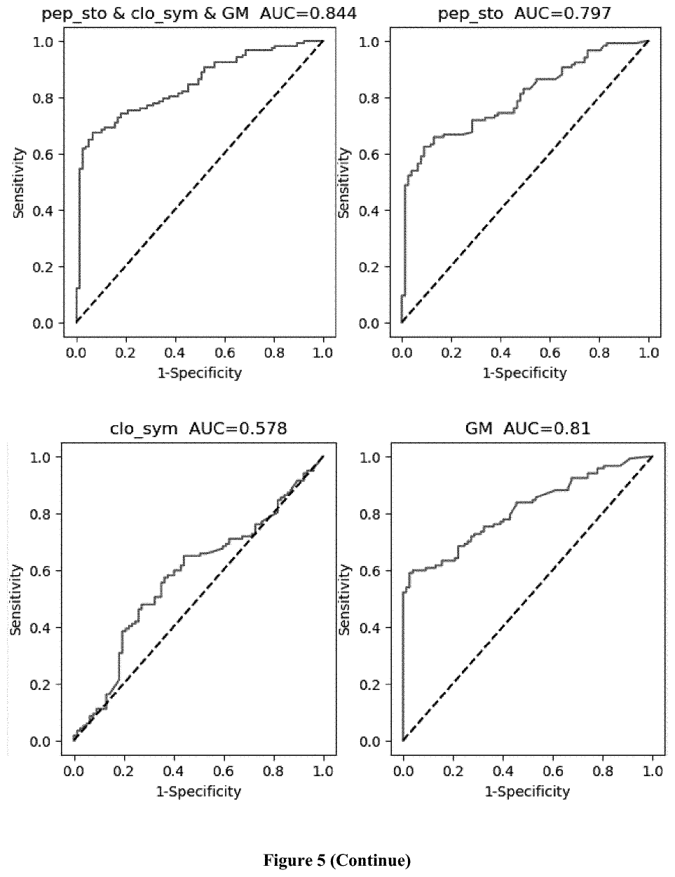

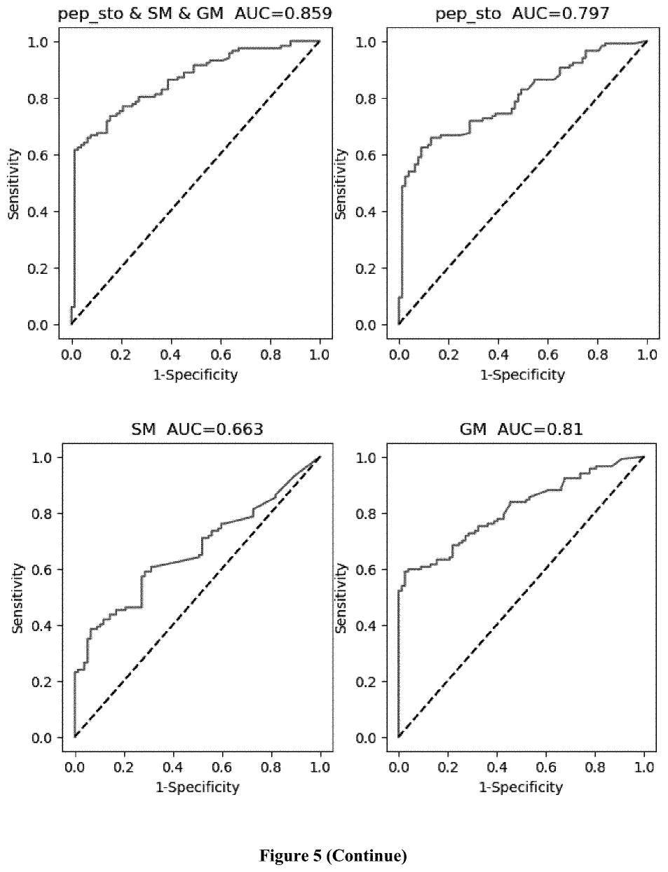

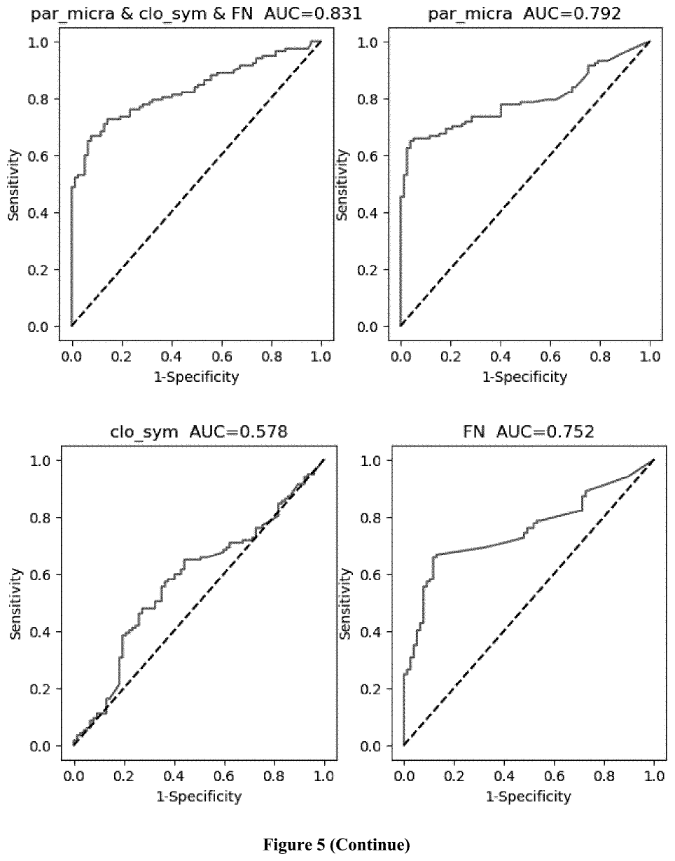

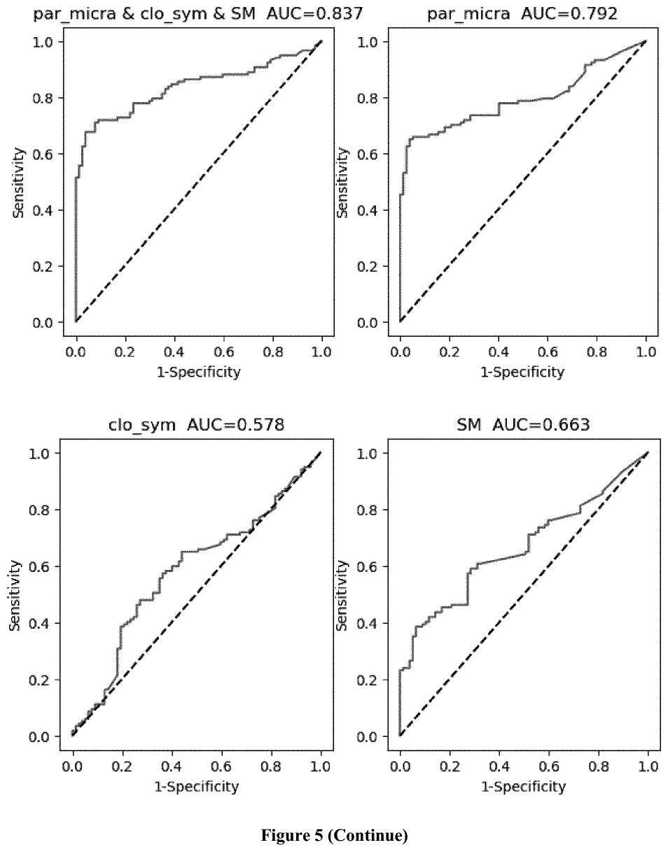

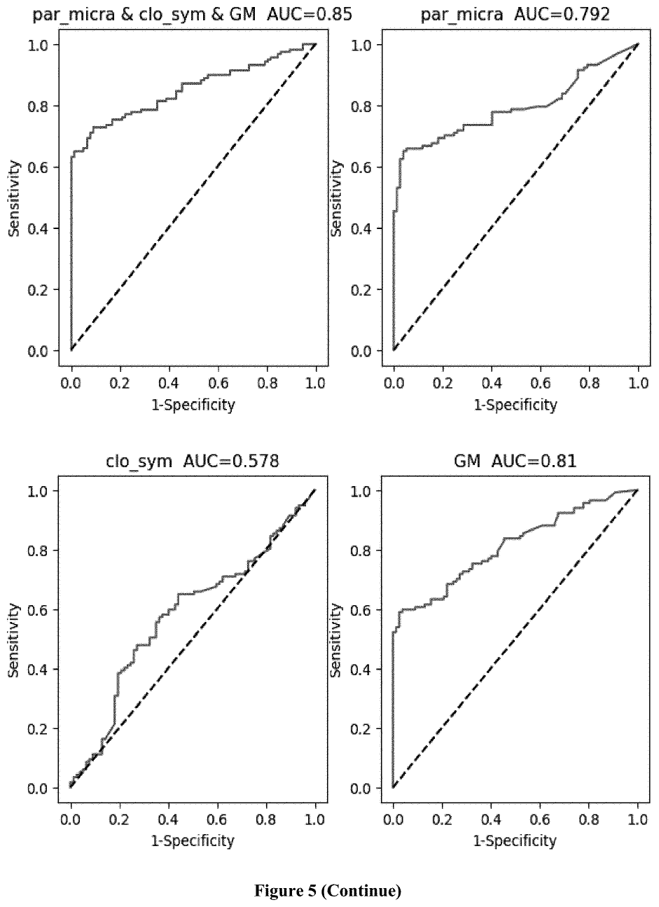

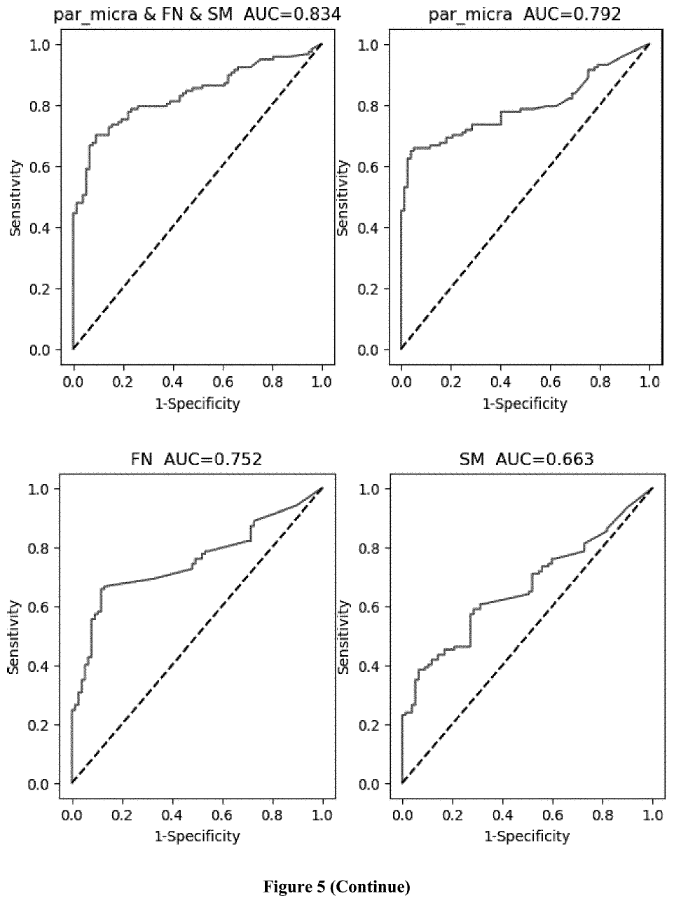

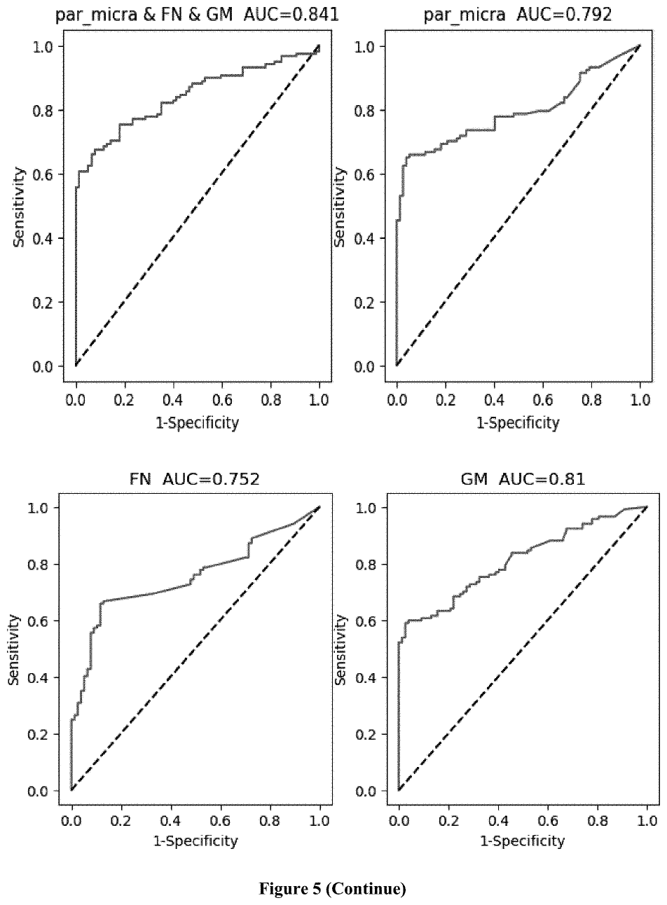

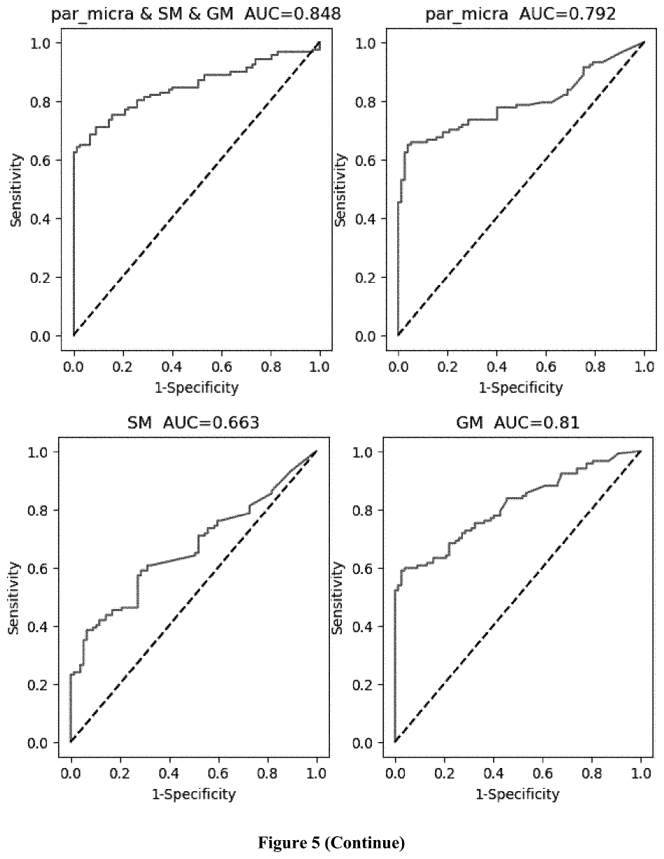

As shown in FIG. 5, the inventors also found that the combination, illustrated in anyone of the following groups (1)-(13), of three bacterial markers demonstrated significantly better results compared to using a single bacterial marker:
(1) Peptostreptococcus stomatis, Parvimonas micra, Clostridium symbiosum;
(2) Peptostreptococcus stomatis, Parvimonas micra, Fusobacterium nucleatum;
(3) Peptostreptococcus stomatis, Parvimonas micra, Solobacterium moorei;
(4) Pptostreptococcus stomatis, Parvimonas micra, Gemella morbillorum;
(5) Peptostreptococcus stomatis, Clostridium symbiosum, Fusobacterium nucleatum;
(6) Peptostreptococcus stomatis, Clostridium symbiosum, Gemella morbillorum;
(7) Peptostreptococcus stomatis, Solobacterium moorei, Gemella morbillorum;
(8) Parvimonas micra, Clostridium symbiosum, Fusobacterium nucleatum;
(9) Parvimonas micra, Clostridium symbiosum, Solobacterium moorei;
(10) Parvimonas micra, Clostridium symbiosum, Gemella morbillorum;
(11) Parvimonas micra, Fusobacterium nucleatum, Solobacterium moorei;
(12) Parvimonas micra, Fusobacterium nucleatum, Gemella morbillorum;
(13) Parvimonas micra, Solobacterium moorei, Gemella morbillorum.

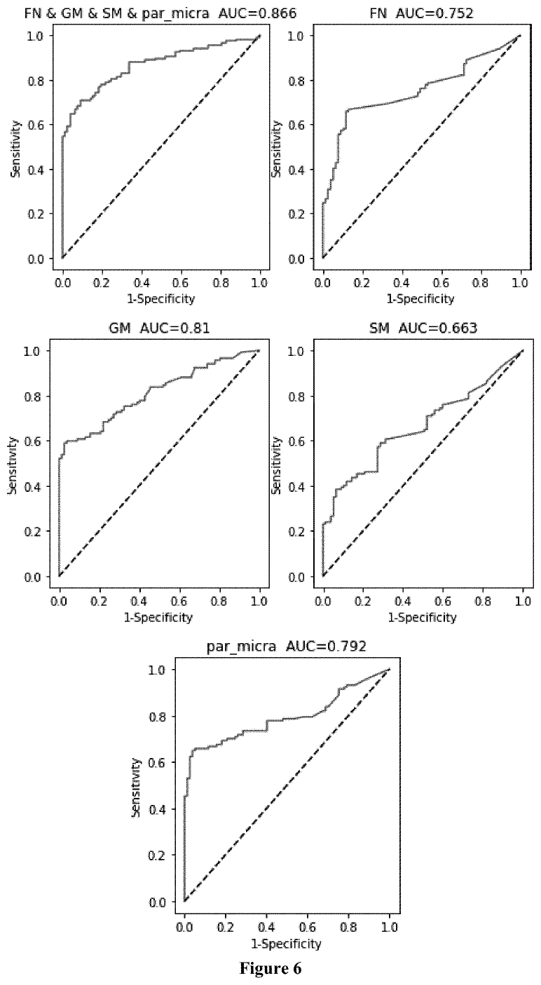

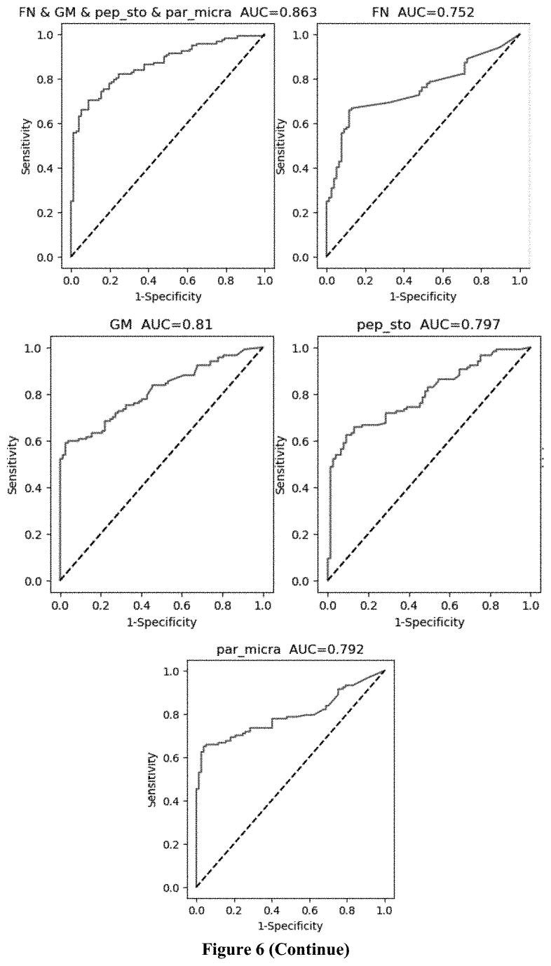

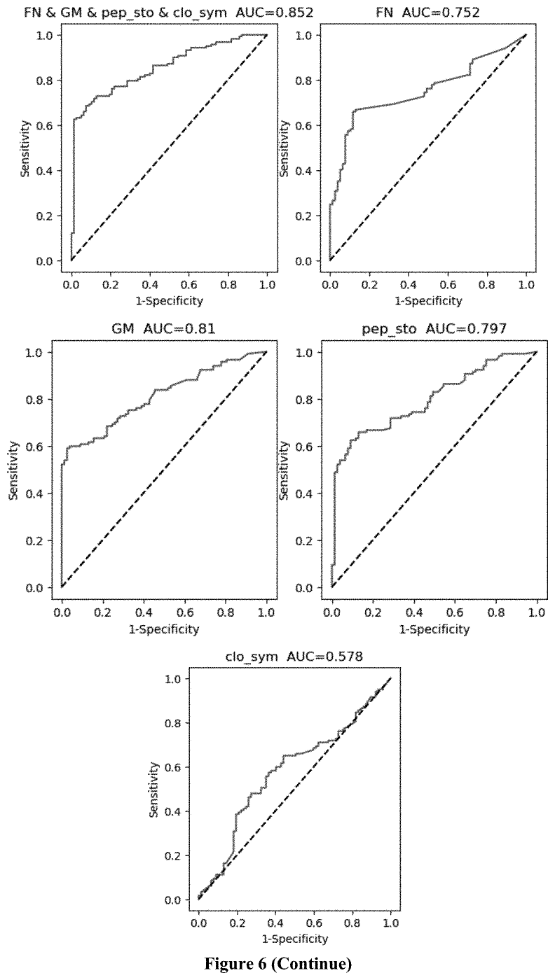

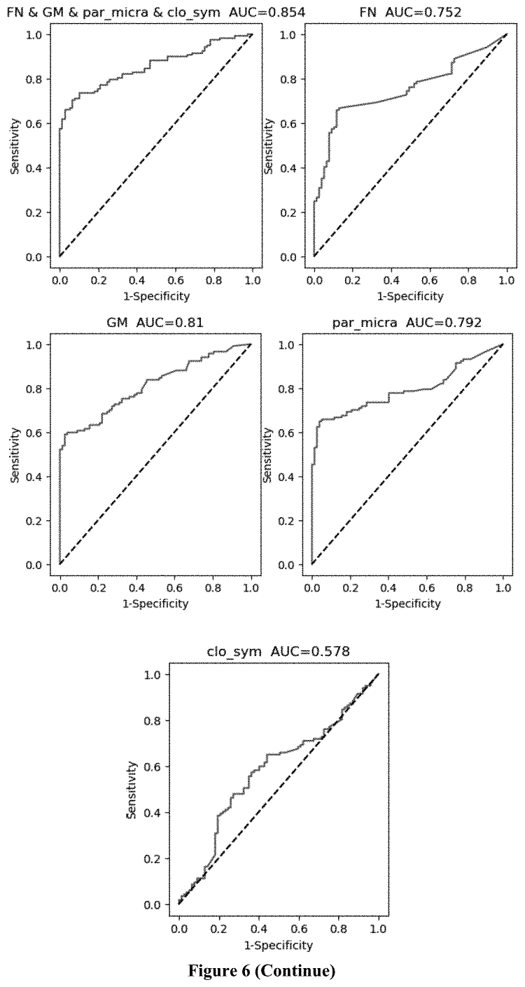

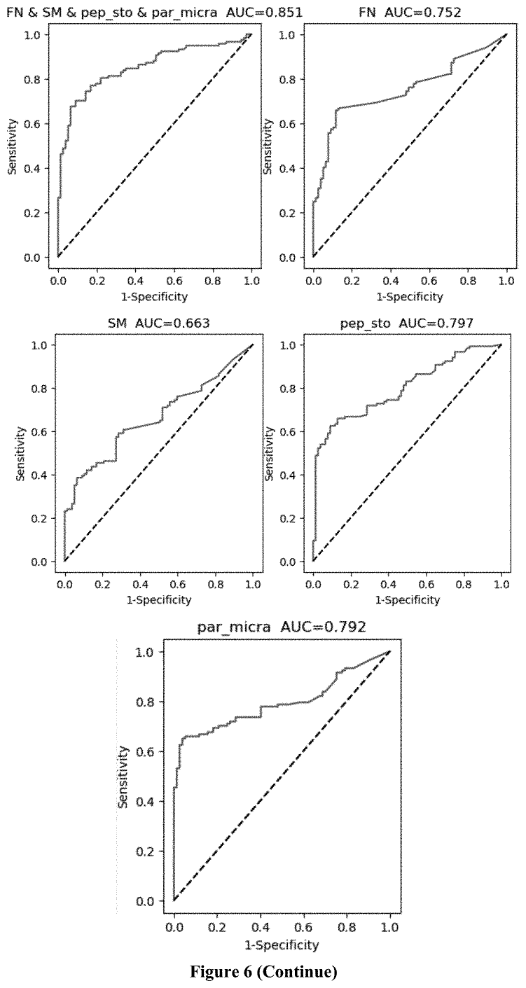

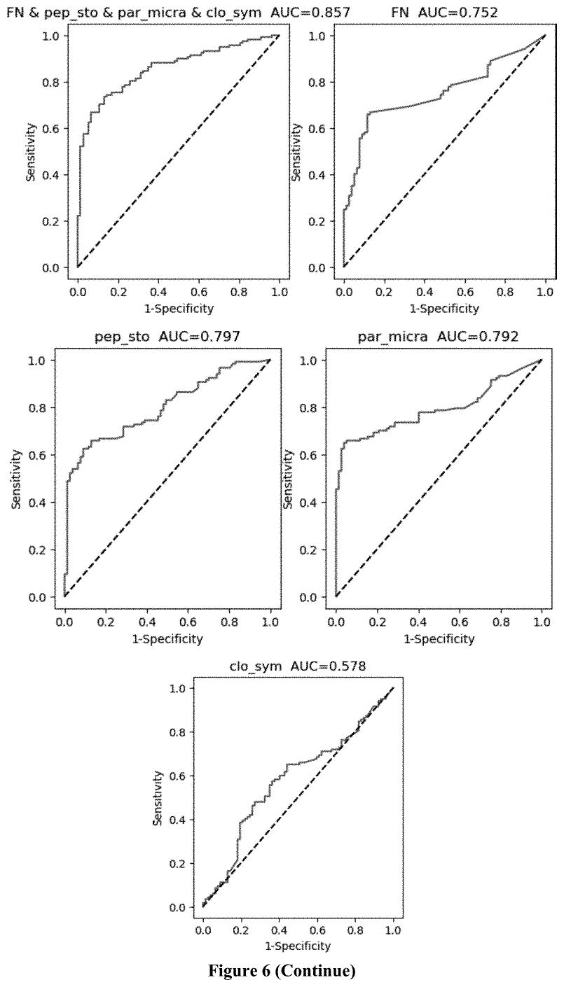

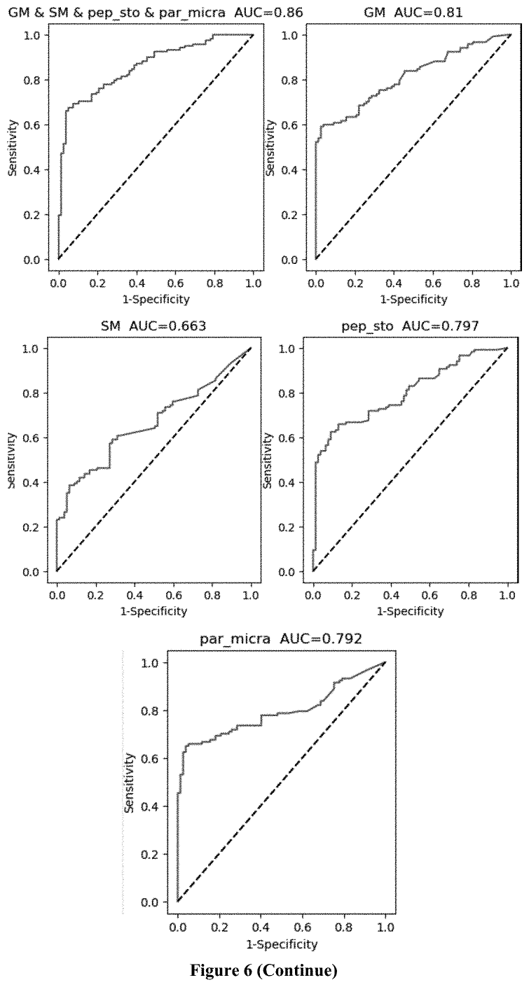

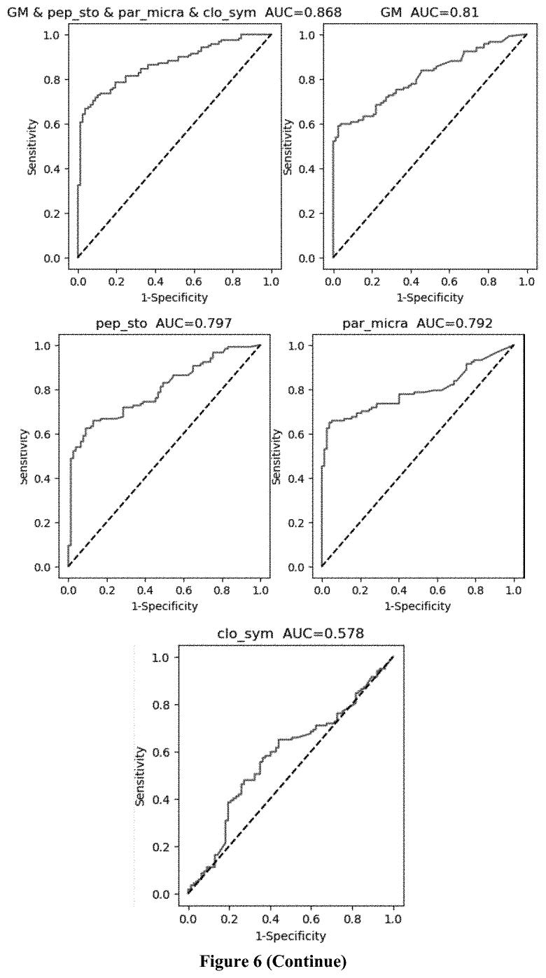

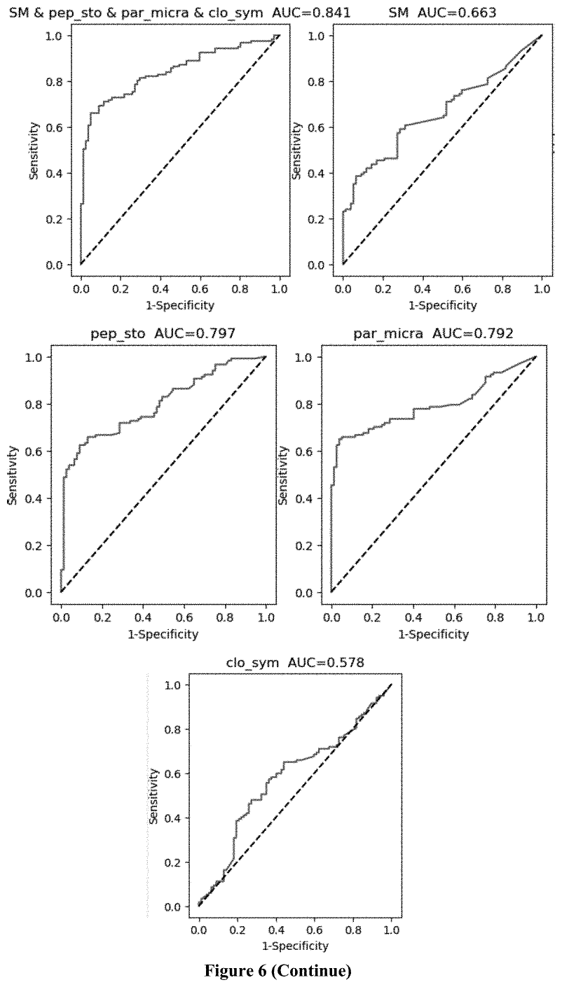

As shown in FIG. 6, the inventors also found that the following combination of four bacterial markers demonstrated significantly better results compared to using a single bacterial marker:
(1) Fusobacterium nucleatum, Gemella morbillorum, Solobacterium moorei, Parvimonas micra; or
(2) Fusobacterium nucleatum, Gemella morbillorum, Peptostreptococcus stomatis, Parvimonas micra; or
(3) Fusobacterium nucleatum, Gemella morbillorum, Peptostreptococcus stomatis, Clostridium symbiosum; or
(4) Fusobacterium nucleatum, Gemella morbillorum, Parvimonas micra, Clostridium symbiosum; or
(5) Fusobacterium nucleatum, Solobacterium moorei, Peptostreptococcus stomatis, Parvimonas micra; or
(6) Fusobacterium nucleatum, Peptostreptococcus stomatis, Parvimonas micra, Clostridium symbiosum; or
(7) Gemella morbillorum, Solobacterium moorei, Parvimonas micra, Clostridium symbiosum; or
(8) Gemella morbillorum, Peptostreptococcus stomatis, Parvimonas micra, Clostridium symbiosum; or
(9) Solobacterium moorei, Peptostreptococcus stomatis, Parvimonas micra, Clostridium symbiosum.

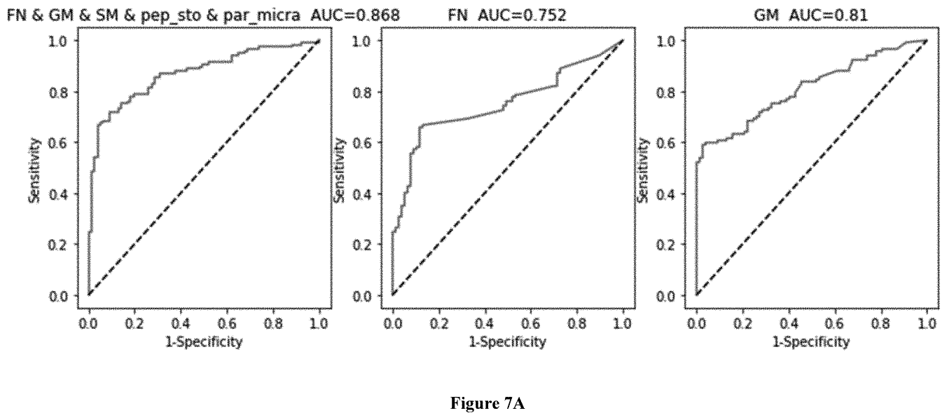

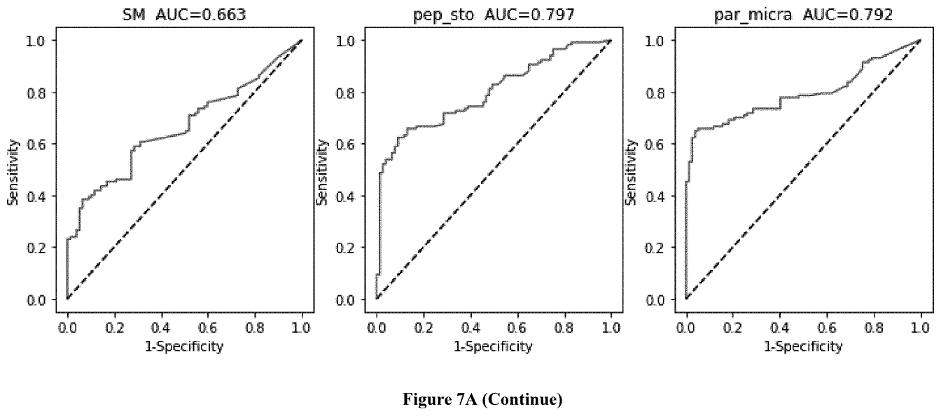

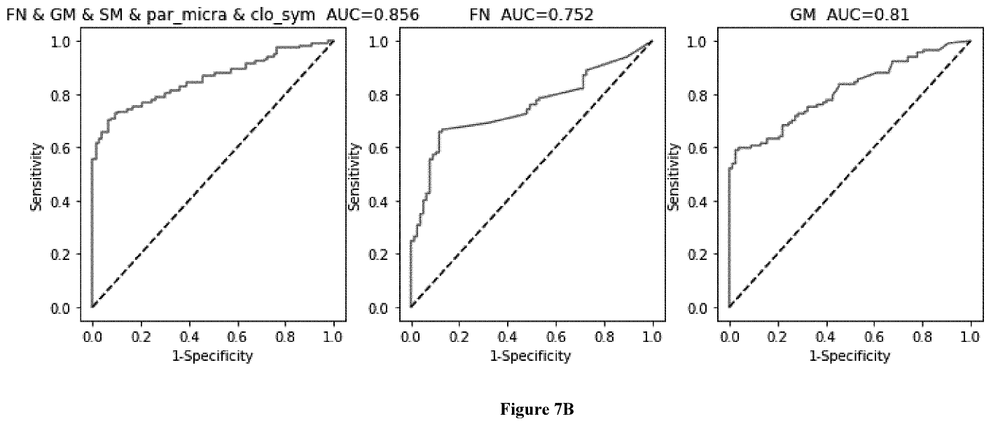

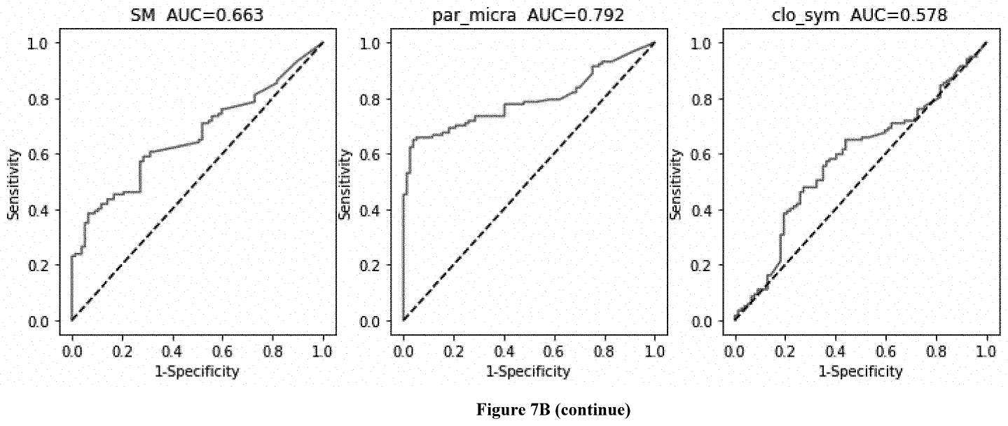

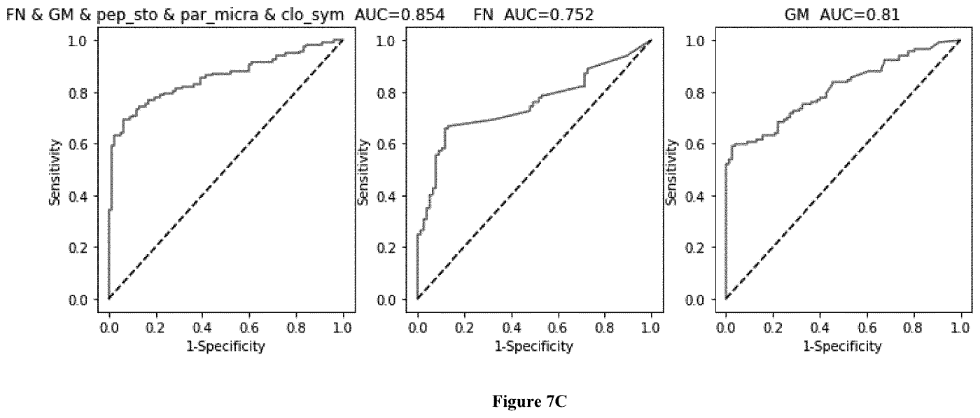

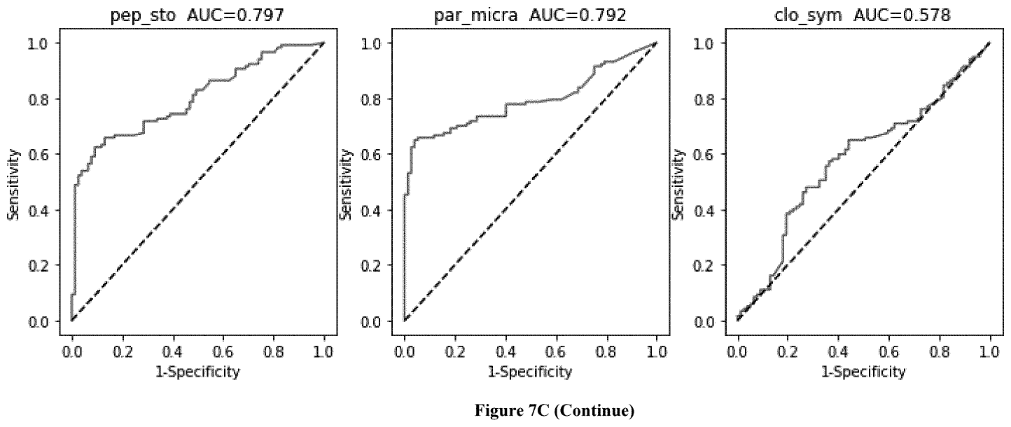

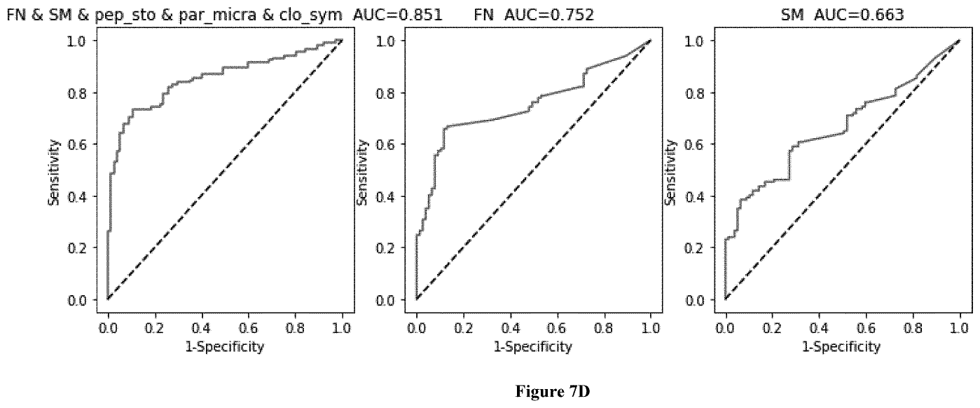

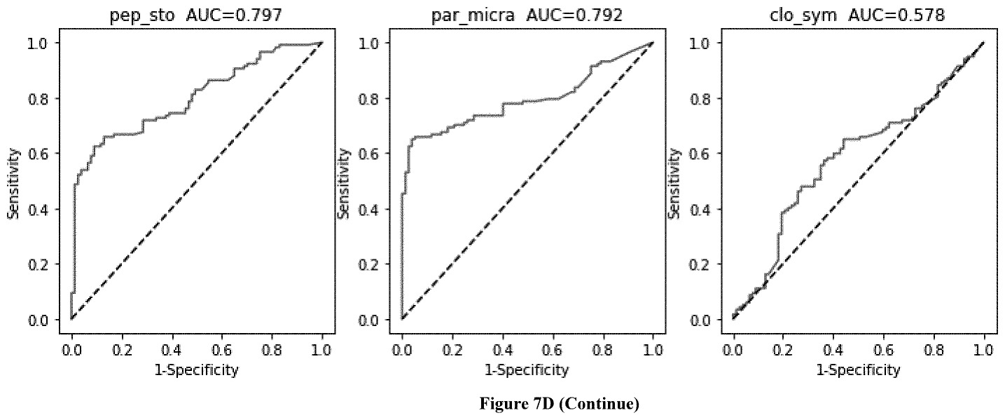

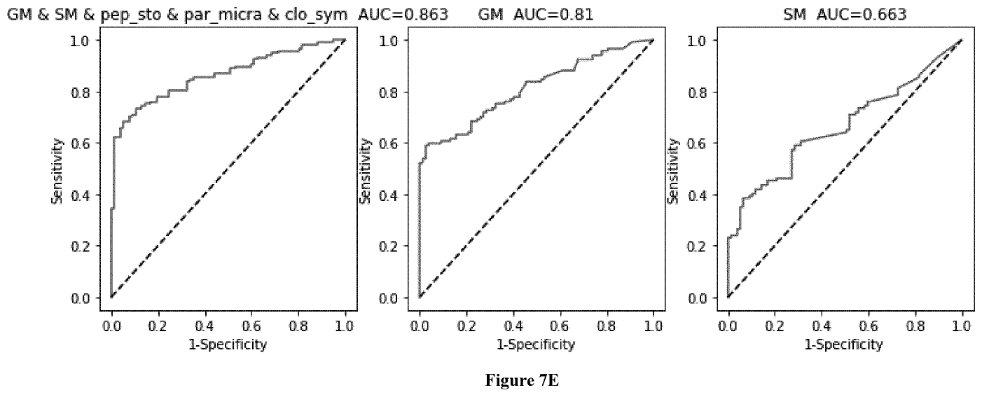

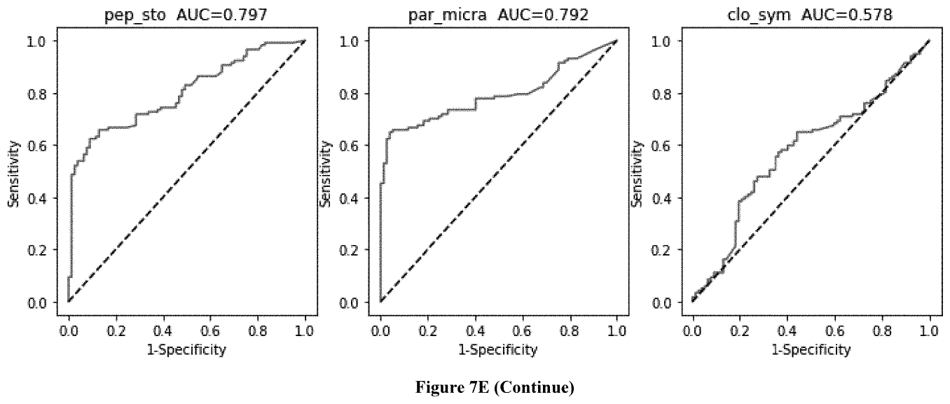

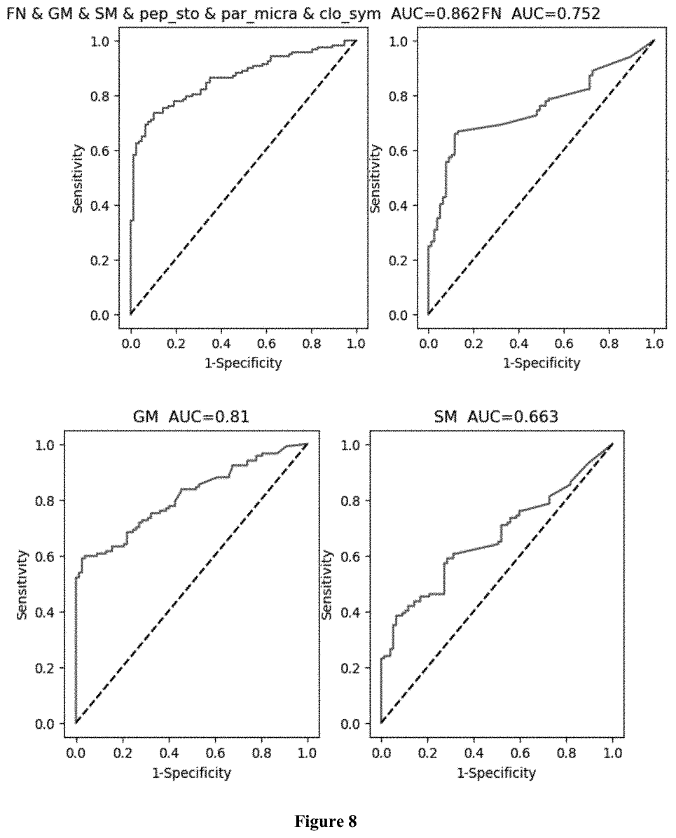

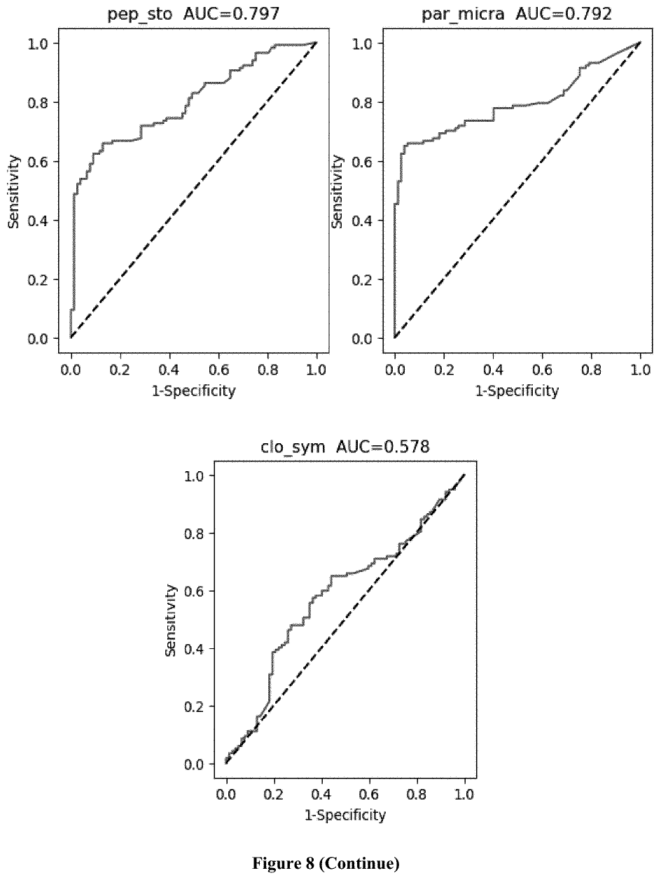

As illustrated in FIGS. 7A-7E and FIG. 8, the inventors also found that the following combination of five or six bacterial markers demonstrated significantly better results compared to using a single bacterial marker:
(1) Fusobacterium nucleatum, Gemella morbillorum, Solobacterium moorei, Peptostreptococcus stomatis, and Parvimonas micro; or
(2) Fusobacterium nucleatum, Gemella morbillorum, Solobacterium moorei, Parvimonas micra and Clostridium symbiosum; or
(3) Fusobacterium nucleatum, Gemella morbillorum, Peptostreptococcus stomatis, Parvimonas micra and Clostridium symbiosum; or
(4) Fusobacterium nucleatum, Solobacterium moorei, Peptostreptococcus stomatis, Parvimonas micra and Clostridium symbiosum; or
(5) Gemella morbillorum, Solobacterium moorei, Peptostreptococcus stomatis, Parvimonas micra and Clostridium symbiosum; or
(6) Fusobacterium nucleatum, Gemella morbillorum, Solobacterium moorei, Peptostreptococcus stomatis, Parvimonas micra and Clostridium symbiosum.

### EXAMPLE 5

This example illustrates the diagnosis of colorectal cancer using a combination of multiple bacterial markers and fecal immunochemical test (FIT).

The analysis used 121 CRC samples and 78 PE samples. The abundance of each bacterial marker (copy number in 100 ng total DNA extracted from the fecal sample) was measured using ddPCR as described above. A logistic regression classifier was trained using 5-fold cross validation to generate hyper-parameters. The logistic regression classifier was then re-trained with the hyper-parameters and used for test prediction. To compare whether the ROC curves generated by two different classifiers are significantly different, p-value was generated using Delong's test.

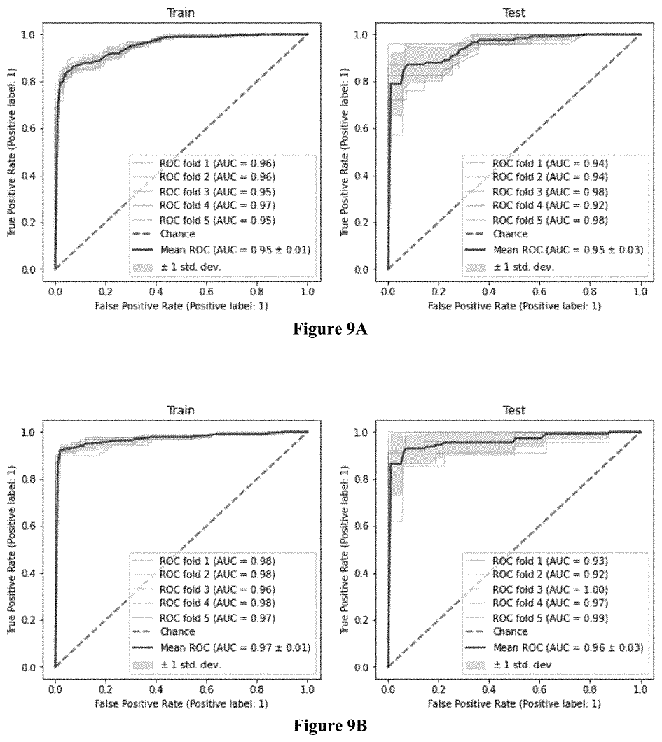

As illustrated in FIGS. 9A and 9B, the combination of bacterial markers and FIT (fecal immunochemical test) resulted in higher sensitivity as compared to FIT. As shown in FIG. 9A and FIG. 9B, when the specificity was 90%, the sensitivity increased from 89.4% in FIT to 95.5% in the combination of FIT and bacterial markers.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

What is claimed is:

1. A method for diagnosing colorectal cancer or advanced colorectal adenoma in a subject, the method comprising:
measuring in a feces sample isolated from the subject levels of at least two bacterial markers selected from the group consisting of Fusobacterium nucleatum, Peptostreptococcus stomatis, Parvimonas micra, Gemella morbillorum, Solobacterium moorei, Porphyromonas asaccharolytica, Peptostreptococcus anaerobius, Hungatella hathewayi, Streptococcus gallolyticus, Clostridium symbiosum, Prevotella copri, Prevotella nigrescens, Bacteroides clarus, genotoxic pks+Escherichia coli and gene bft from Bacteroides fragilis, and
evaluating the measured levels of the bacterial markers,
determining that the subject is healthy or has colorectal cancer or advanced colorectal adenoma.

2. The method of claim 1, comprising measuring levels of at least two bacterial markers selected from the group consisting of Fusobacterium nucleatum, Peptostreptococcus stomatis, Parvimonas micra, Gemella morbillorum, Solobacterium moorei, and Clostridium symbiosum;
preferably, the method comprising measuring levels of
(1) Peptostreptococcus stomatis and Parvimonas micra; or
(2) Peptostreptococcus stomatis and Fusobacterium nucleatum; or
(3) Peptostreptococcus stomatis and Gemella morbillorum; or
(4) Fusobacterium nucleatum and Parvimonas micra; or
(5) Gemella morbillorum and Parvimonas micra; or
(6) Fusobacterium nucleatum and Gemella morbillorum.

3. The method of claim 1, comprising measuring levels of at least three bacterial markers selected from the group consisting of Fusobacterium nucleatum, Peptostreptococcus stomatis, Parvimonas micra, Gemella morbillorum, Solobacterium moorei, and Clostridium symbiosum;
preferably, the method comprising measuring levels of
(1) Peptostreptococcus stomatis, Parvimonas micra, Clostridium symbiosum; or
(2) Peptostreptococcus stomatis, Parvimonas micra, Fusobacterium nucleatum; or
(3) Peptostreptococcus stomatis, Parvimonas micra, Solobacterium moorei; or
(4) Peptostreptococcus stomatis, Parvimonas micra, Gemella morbillorum; or
(5) Peptostreptococcus stomatis, Clostridium symbiosum, Fusobacterium nucleatum; or
(6) Peptostreptococcus stomatis, Clostridium symbiosum, Gemella morbillorum; or
(7) Peptostreptococcus stomatis, Solobacterium moorei, Gemella morbillorum; or
(8) Parvimonas micra, Clostridium symbiosum, Fusobacterium nucleatum; or
(9) Parvimonas micra, Clostridium symbiosum, Solobacterium moorei; or
(10) Parvimonas micra, Clostridium symbiosum, Gemella morbillorum; or
(11) Parvimonas micra, Fusobacterium nucleatum, Solobacterium moorei; or
(12) Parvimonas micra, Fusobacterium nucleatum, Gemella morbillorum; or
(13) Parvimonas micra, Solobacterium moorei, Gemella morbillorum.

4. The method of claim 1, comprising measuring levels of at least four bacterial markers selected from the group consisting of Fusobacterium nucleatum, Peptostreptococcus stomatis, Parvimonas micra, Gemella morbillorum, Solobacterium moorei, and Clostridium symbiosum;
preferably, the method comprising measuring levels of
(1) Fusobacterium nucleatum, Gemella morbillorum, Solobacterium moorei, Parvimonas micra; or
(2) Fusobacterium nucleatum, Gemella morbillorum, Peptostreptococcus stomatis, Parvimonas micra; or
(3) Fusobacterium nucleatum, Gemella morbillorum, Peptostreptococcus stomatis, Clostridium symbiosum; or
(4) Fusobacterium nucleatum, Gemella morbillorum, Parvimonas micra, Clostridium symbiosum; or
(5) Fusobacterium nucleatum, Solobacterium moorei, Peptostreptococcus stomatis, Parvimonas micra; or
(6) Fusobacterium nucleatum, Peptostreptococcus stomatis, Parvimonas micra, Clostridium symbiosum; or
(7) Gemella morbillorum, Solobacterium moorei, Parvimonas micra, Clostridium symbiosum; or
(8) Gemella morbillorum, Peptostreptococcus stomatis, Parvimonas micra, Clostridium symbiosum; or
(9) Solobacterium moorei, Peptostreptococcus stomatis, Parvimonas micra, Clostridium symbiosum.

5. The method of claim 1, comprising measuring levels of at least five bacterial markers selected from the group consisting of Fusobacterium nucleatum, Peptostreptococcus stomatis, Parvimonas micra, Gemella morbillorum, Solobacterium moorei, and Clostridium symbiosum;
preferably, the method comprising measuring levels of
(1) Fusobacterium nucleatum, Gemella morbillorum, Solobacterium moorei, Peptostreptococcus stomatis, and Parvimonas micra; or
(2) Fusobacterium nucleatum, Gemella morbillorum, Solobacterium moorei, Parvimonas micra and Clostridium symbiosum; or
(3) Fusobacterium nucleatum, Gemella morbillorum, Peptostreptococcus stomatis, Parvimonas micra and Clostridium symbiosum; or
(4) Fusobacterium nucleatum, Solobacterium moorei, Peptostreptococcus stomatis, Parvimonas micra and Clostridium symbiosum; or
(5) Gemella morbillorum, Solobacterium moorei, Peptostreptococcus stomatis, Parvimonas micra and Clostridium symbiosum; or
(6) Fusobacterium nucleatum, Gemella morbillorum, Solobacterium moorei, Peptostreptococcus stomatis, Parvimonas micra and Clostridium symbiosum.

6. The method of claim 1, wherein measuring the levels of the bacterial markers comprising detecting a sequence selected from SEQ ID NOs: 4, 8, 12, 16, 20, 24, 28, 32, 36, 40, 44, 48, 52, 56, and 60.

7. A kit of diagnosing colorectal cancer or advanced colorectal adenoma, comprising primers for detecting in a feces sample levels of at least two bacterial markers selected from the group according to the group listed in claim 1.

8. The kit of claim 7, wherein the primers are capable of detecting the levels of at least two bacterial markers selected from the group consisting of Fusobacterium nucleatum, Peptostreptococcus stomatis, Parvimonas micra, Gemella morbillorum, Solobacterium moorei, and Clostridium symbiosum;
preferably, the method comprising measuring levels of
(1) Peptostreptococcus stomatis and Parvimonas micra; or
(2) Peptostreptococcus stomatis and Fusobacterium nucleatum; or
(3) Peptostreptococcus stomatis and Gemella morbillorum; or
(4) Fusobacterium nucleatum and Parvimonas micra; or
(5) Gemella morbillorum and Parvimonas micra; or
(6) Fusobacterium nucleatum and Gemella morbillorum.

9. The kit of claims 7, wherein the primers are capable of detecting the levels of at least three bacterial markers selected from the group consisting of Fusobacterium nucleatum, Peptostreptococcus stomatis, Parvimonas micra, Gemella morbillorum, Solobacterium moorei, and Clostridium symbiosum;
preferably, the method comprising measuring levels of
(1) Peptostreptococcus stomatis, Parvimonas micra, Clostridium symbiosum; or
(2) Peptostreptococcus stomatis, Parvimonas micra, Fusobacterium nucleatum; or
(3) Peptostreptococcus stomatis, Parvimonas micra, Solobacterium moorei; or
(4) Peptostreptococcus stomatis, Parvimonas micra, Gemella morbillorum; or
(5) Peptostreptococcus stomatis, Clostridium symbiosum, Fusobacterium nucleatum; or
(6) Peptostreptococcus stomatis, Clostridium symbiosum, Gemella morbillorum; or
(7) Peptostreptococcus stomatis, Solobacterium moorei, Gemella morbillorum; or
(8) Parvimonas micra, Clostridium symbiosum, Fusobacterium nucleatum; or
(9) Parvimonas micra, Clostridium symbiosum, Solobacterium moorei; or
(10) Parvimonas micra, Clostridium symbiosum, Gemella morbillorum; or
(11) Parvimonas micra, Fusobacterium nucleatum, Solobacterium moorei; or
(12) Parvimonas micra, Fusobacterium nucleatum, Gemella morbillorum; or
(13) Parvimonas micra, Solobacterium moorei, Gemella morbillorum.

10. The kit of claim 7, wherein the primers are capable of detecting the levels of at least four bacterial markers selected from the group consisting of Fusobacterium nucleatum, Peptostreptococcus stomatis, Parvimonas micra, Gemella morbillorum, Solobacterium moorei, and Clostridium symbiosum;
preferably, the method comprising measuring levels of
(1) Fusobacterium nucleatum, Gemella morbillorum, Solobacterium moorei, Parvimonas micra; or
(2) Fusobacterium nucleatum, Gemella morbillorum, Peptostreptococcus stomatis, Parvimonas micra; or
(3) Fusobacterium nucleatum, Gemella morbillorum, Peptostreptococcus stomatis, Clostridium symbiosum; or
(4) Fusobacterium nucleatum, Gemella morbillorum, Parvimonas micra, Clostridium symbiosum; or
(5) Fusobacterium nucleatum, Solobacterium moorei, Peptostreptococcus stomatis, Parvimonas micra; or
(6) Fusobacterium nucleatum, Peptostreptococcus stomatis, Parvimonas micra, Clostridium symbiosum; or
(7) Gemella morbillorum, Solobacterium moorei, Parvimonas micra, Clostridium symbiosum; or
(8) Gemella morbillorum, Peptostreptococcus stomatis, Parvimonas micra, Clostridium symbiosum; or
(9) Solobacterium moorei, Peptostreptococcus stomatis, Parvimonas micra, Clostridium symbiosum.

11. The kit of claim 7, wherein the primers are capable of detecting the levels of at least five bacterial markers selected from the group consisting of Fusobacterium nucleatum, Peptostreptococcus stomatis, Parvimonas micra, Gemella morbillorum, Solobacterium moorei, and Clostridium symbiosum;
preferably, the method comprising measuring levels of
(1) Fusobacterium nucleatum, Gemella morbillorum, Solobacterium moorei, Peptostreptococcus stomatis, and Parvimonas micra; or
(2) Fusobacterium nucleatum, Gemella morbillorum, Solobacterium moorei, Parvimonas micra and Clostridium symbiosum; or
(3) Fusobacterium nucleatum, Gemella morbillorum, Peptostreptococcus stomatis, Parvimonas micra and Clostridium symbiosum; or
(4) Fusobacterium nucleatum, Solobacterium moorei, Peptostreptococcus stomatis, Parvimonas micra and Clostridium symbiosum; or
(5) Gemella morbillorum, Solobacterium moorei, Peptostreptococcus stomatis, Parvimonas micra and Clostridium symbiosum; or
(6) Fusobacterium nucleatum, Gemella morbillorum, Solobacterium moorei, Peptostreptococcus stomatis, Parvimonas micra and Clostridium symbiosum.

12. A method for treating colorectal cancer or advanced colorectal adenoma in a subject, the method comprising:
administering to the subject a therapeutically effective amount of a drug useful for treating colorectal cancer or advanced colorectal adenoma,
wherein the subject has been determined to have colorectal cancer or adenoma by a machine learning classifier based on levels of at least two bacterial markers measured in a feces sample isolated form the subject, wherein the at least bacterial markers are selected from the group according to the group listed in claim 1.

13. The method of claim 12, wherein the subject has been determined to have colorectal cancer or advanced colorectal adenoma by the machine learning classifier based on levels of at least two bacterial markers selected from the group consisting of Fusobacterium nucleatum, Peptostreptococcus stomatis, Parvimonas micra, Gemella morbillorum, Solobacterium moorei, and Clostridium symbiosum.

14. A agent for use in manufacturing a kit of diagnosing colorectal cancer or advanced colorectal adenoma, said agent is capable of measuring in a feces sample levels of at least two bacterial markers selected from the group according to the group listed in claim 1.

15. A computer-implemented method for identifying a discriminative region within a group of sequences, the method comprising:
obtaining a plurality of sequences comprising
a group of target sequences for identifying a discriminative region within the group, and
a group of background sequences;
decomposing each sequence within the group of target sequences into overlapping kmers, wherein each kmer has a length of 4 to 31;
identifying a pair of kmers, wherein
the pair of kmers occurs at most once in each sequence within the group of target polynucleotide sequences,
the pair of kmers has a distance ranging from 20 to 1000,
the pair of kmers are not identical, and
the pair of kmers occur more than a threshold number of the target sequences;
retrieving all regions flanged by the pair of kmers in the target sequences;
aligning the regions retrieved to determine that the regions are conserved;
generating a consensus sequence based on the regions retrieved;
determining that the consensus sequence does not occur in the group of background sequences; and
retaining the consensus sequence as a discriminative region for the group of target sequences.

16. The method of claim 15, wherein the group of target polynucleotide sequences are genomic sequences of a bacterial species or a viral species; preferably, the bacterial species is a gut microbial species, and the viral species is HIV, HCV, or Covid-19.

17. The method of claim 15, further comprising designing a pair of primers for amplifying the discriminative region.

18. The method of claim 15, comprising filtering the kmers before the step of identifying the pair of kmers according to a criterion selected from:
the kmer occurs less than or more than a threshold percentage of the target sequences;
the kmer has a homopolymer, dimer or trimer of more than a threshold; and
the kmer has a GC content more than or less than a threshold.

19. A non-transitory computer readable medium having instructions stored thereon, wherein the instructions, when executed by a processor, cause the processor to perform the method of claim 15.

20. A bacterial marker set for use in diagnosing colorectal cancer or advanced colorectal adenoma comprising at least two sequences selected from the group consisting of SEQ ID NOs: 4, 8, 12, 16, 20, 24, 28, 32, 36, 40, 44, 48, 52, 56, and 60.


SEQUENCE LISTING (60 sequences)

SEQ ID NO: 1 - DNA, 21 bp, synthetic construct
  source 1..21: mol_type = other DNA
agttcggttcacttacaccag

SEQ ID NO: 2 - DNA, 21 bp, synthetic construct
  source 1..21: mol_type = other DNA
tggtcctgtagttgcttcaac

SEQ ID NO: 3 - DNA, 26 bp, synthetic construct
  source 1..26: mol_type = other DNA
tcacccagaatacaagcacacagttg

SEQ ID NO: 4 - DNA, 71 bp, Peptostreptococcus anaerobius
  source 1..71: mol_type = genomic DNA
agttcggttcacttacaccaggtcacccagaatacaagcacacagttggtgttgaagcaactacaggacca

SEQ ID NO: 5 - DNA, 20 bp, synthetic construct
  source 1..20: mol_type = other DNA
ctgccgctagtttcaaacac

SEQ ID NO: 6 - DNA, 21 bp, synthetic construct
  source 1..21: mol_type = other DNA
tcttcaatgtgtcgctcagag

SEQ ID NO: 7 - DNA, 22 bp, synthetic construct
  source 1..22: mol_type = other DNA
ctgccggtgcagctatcggctt

SEQ ID NO: 8 - DNA, 72 bp, Prevotella copri
  source 1..72: mol_type = genomic DNA
ctgccgctagtttcaaacacgtttctcctgccggtgcagctatcggcttgcctctgagcgacacattgaaga

SEQ ID NO: 9 - DNA, 21 bp, synthetic construct
  source 1..21: mol_type = other DNA
gcatcctacctaccaatagcc

SEQ ID NO: 10 - DNA, 20 bp, synthetic construct
  source 1..20: mol_type = other DNA
cgtctatatgccccaactcc

SEQ ID NO: 11 - DNA, 26 bp, synthetic construct
  source 1..26: mol_type = other DNA
tatgccggtcttggccatgcattcca

SEQ ID NO: 12 - DNA, 85 bp, Peptostreptococcus stomatis
  source 1..85: mol_type = genomic DNA
gcatcctacctaccaatagccatagtggatgtaggtatgccggtcttggccatgcattccataagggagttggggcatatagacg

SEQ ID NO: 13 - DNA, 20 bp, synthetic construct
  source 1..20: mol_type = other DNA
tcttccgcctgtgttattcc

SEQ ID NO: 14 - DNA, 21 bp, synthetic construct
  source 1..21: mol_type = other DNA
cagcttcaccatcacaatgac

SEQ ID NO: 15 - DNA, 22 bp, synthetic construct
  source 1..22: mol_type = other DNA
ccgcagctctgccagattgtgg

SEQ ID NO: 16 - DNA, 86 bp, Polyketide synthetase
  source 1..86: mol_type = genomic DNA
tcttccgcctgtgttattccccgcagctctgccagattgtggatatcaacaatatggcgatagcggtcattgtgatggtgaagctg

SEQ ID NO: 17 - DNA, 20 bp, synthetic construct
  source 1..20: mol_type = other DNA
tgcgtccttgtatcgttagg

SEQ ID NO: 18 - DNA, 21 bp, synthetic construct
  source 1..21: mol_type = other DNA
gtgagtgatcaggtctccatc

SEQ ID NO: 19 - DNA, 24 bp, synthetic construct
  source 1..24: mol_type = other DNA
tcacctgccatagcgattgcgtct

SEQ ID NO: 20 - DNA, 67 bp, Porphyromonas asaccharolytica
  source 1..67: mol_type = genomic DNA
tgcgtccttgtatcgttaggtcacctgccatagcgattgcgtctaagatggagacctgatcactcac

SEQ ID NO: 21 - DNA, 21 bp, synthetic construct
  source 1..21: mol_type = other DNA
agcgactaagcaacatcttcc

SEQ ID NO: 22 - DNA, 21 bp, synthetic construct
  source 1..21: mol_type = other DNA
tcagaaaggagctgttatggc

SEQ ID NO: 23 - DNA, 24 bp, synthetic construct
  source 1..24: mol_type = other DNA
cgatatacgctcttgttgattctc

SEQ ID NO: 24 - DNA, 77 bp, Streptococcus gallolyticus
  source 1..77: mol_type = genomic DNA
agcgactaagcaacatcttccgccgttcgcgcgatatacgctcttgttgattctcagccataacagctcctttctga

SEQ ID NO: 25 - DNA, 21 bp, synthetic construct
  source 1..21: mol_type = other DNA
agagcttccatttgagaaggc

SEQ ID NO: 26 - DNA, 21 bp, synthetic construct
  source 1..21: mol_type = other DNA
acctttctctgcttgtatggc

SEQ ID NO: 27 - DNA, 20 bp, synthetic construct
  source 1..20: mol_type = other DNA
cctcatcaccgacgatcaca

SEQ ID NO: 28 - DNA, 75 bp, Hungatella hathewayi
  source 1..75: mol_type = genomic DNA
agagcttccatttgagaaggccggtcttccggtcctcatcaccgacgatcacaagccatacaagcagagaaaggt

SEQ ID NO: 29 - DNA, 22 bp, synthetic construct
  source 1..22: mol_type = other DNA
cgtagctattatttgctgtgcg

SEQ ID NO: 30 - DNA, 23 bp, synthetic construct
  source 1..23: mol_type = other DNA
tgtgtattattacccctccttgc

SEQ ID NO: 31 - DNA, 24 bp, synthetic construct
  source 1..24: mol_type = other DNA
acgcccctacacttgcaacgaact

SEQ ID NO: 32 - DNA, 82 bp, Prevotella nigrescens
  source 1..82: mol_type = genomic DNA
cgtagctattatttgctgtgcgagcttagaagaggacgcccctacacttgcaacgaactgcaaggaggggtaataatacaca

SEQ ID NO: 33 - DNA, 22 bp, synthetic construct
  source 1..22: mol_type = other DNA
gcatacataacccattcatgcc

SEQ ID NO: 34 - DNA, 23 bp, synthetic construct
  source 1..23: mol_type = other DNA
gaaacagttcttgacaactcacc

SEQ ID NO: 35 - DNA, 34 bp, synthetic construct
  source 1..34: mol_type = other DNA
ctggataggattttccgttaattgcataataagc

SEQ ID NO: 36 - DNA, 92 bp, Parvimonas micra
  source 1..92: mol_type = genomic DNA
gcatacataacccattcatgccatccttctggataggattttccgttaattgcataataagcttgaattggtgagttgtcaagaactgtttc

SEQ ID NO: 37 - DNA, 20 bp, synthetic construct
  source 1..20: mol_type = other DNA
tgaatatagttgcccgagcg

SEQ ID NO: 38 - DNA, 21 bp, synthetic construct
  source 1..21: mol_type = other DNA
accttctgctttcggattagc

SEQ ID NO: 39 - DNA, 28 bp, synthetic construct
  source 1..28: mol_type = other DNA
agggctggatggctttactgcttcacta

SEQ ID NO: 40 - DNA, 85 bp, Enterotoxigenic bacteroides fragilis
  source 1..85: mol_type = genomic DNA
tgaatatagttgcccgagcggcaatgccgatgaagggctggatggctttactgcttcactaaaagctaatccgaaagcagaaggt

SEQ ID NO: 41 - DNA, 22 bp, synthetic construct
  source 1..22: mol_type = other DNA
agcaatacttccccatacttgg

SEQ ID NO: 42 - DNA, 20 bp, synthetic construct
  source 1..20: mol_type = other DNA
actttccgttcagagttcgg

SEQ ID NO: 43 - DNA, 24 bp, synthetic construct
  source 1..24: mol_type = other DNA
tcggtattgtcaaacttccagccg

SEQ ID NO: 44 - DNA, 69 bp, Bacteroides clarus
  source 1..69: mol_type = genomic DNA
agcaatacttccccatacttggtcggtattgtcaaacttccagccgtatccgaactctgaacggaaagt

SEQ ID NO: 45 - DNA, 21 bp, synthetic construct
  source 1..21: mol_type = other DNA
tgggtcagtttggatttcagg

SEQ ID NO: 46 - DNA, 22 bp, synthetic construct
  source 1..22: mol_type = other DNA
ccgttattgatcagtgcaaagg

SEQ ID NO: 47 - DNA, 26 bp, synthetic construct
  source 1..26: mol_type = other DNA
ccctccggtgataccagctcgactgt

SEQ ID NO: 48 - DNA, 82 bp, Clostridium symbiosum
  source 1..82: mol_type = genomic DNA
tgggtcagtttggatttcaggttgatgcgatctccctccggtgataccagctcgactgttcctttgcactgatcaataacgg

SEQ ID NO: 49 - DNA, 20 bp, synthetic construct
  source 1..20: mol_type = other DNA
caaggtgctgatgttgttgc

SEQ ID NO: 50 - DNA, 21 bp, synthetic construct
  source 1..21: mol_type = other DNA
cgaaaccttctgttgttgctc

SEQ ID NO: 51 - DNA, 21 bp, synthetic construct
  source 1..21: mol_type = other DNA
acaggacaatctctactagac

SEQ ID NO: 52 - DNA, 88 bp, Fusobacterium nucleatum
  source 1..88: mol_type = genomic DNA
caaggtgctgatgttgttgcagttataagaacaacaggacaatctctactagacttcgtaccttatggagcaacaacagaaggtttcg

SEQ ID NO: 53 - DNA, 21 bp, synthetic construct
  source 1..21: mol_type = other DNA
tgcataacgagattacacctg

SEQ ID NO: 54 - DNA, 22 bp, synthetic construct
  source 1..22: mol_type = other DNA
cactttacatcttcccaaacac

SEQ ID NO: 55 - DNA, 28 bp, synthetic construct
  source 1..28: mol_type = other DNA
cacgtcgtaatgttttgaccaatgcact

SEQ ID NO: 56 - DNA, 92 bp, Solobacterium moorei
  source 1..92: mol_type = genomic DNA
atgcataacgagattacacctgaggaagccaaaacattcccacgtcgtaatgttttgaccaatgcactaggtgtttgggaagatgtaaagtg

SEQ ID NO: 57 - DNA, 22 bp, synthetic construct
  source 1..22: mol_type = other DNA
cgtgaaactgcttatcgcttag

SEQ ID NO: 58 - DNA, 22 bp, synthetic construct
  source 1..22: mol_type = other DNA
agttttcctggatgttctcctg

SEQ ID NO: 59 - DNA, 28 bp, synthetic construct
  source 1..28: mol_type = other DNA
taatcggggctgcaaaaatggtactaga

SEQ ID NO: 60 - DNA, 86 bp, Gemella morbillorum
  source 1..86: mol_type = genomic DNA
cgtgaaactgcttatcgcttagcggcacatacagtaatcggggctgcaaaaatggtactagaaacaggagaacatccaggaaaact